(19) United States
(12) Patent Application Publication
(10) Pub. No.: US 2007/0049897 A1
(43) Pub. Date: Mar. 1, 2007

(54) Disposable pull-on garment having frangible belt

(76) Inventors: Gary Dean LaVon, Liberty Township, OH (US); Stephen Joseph Lange, Wyoming, OH (US); Kara Marie Cain, Cincinnati, OH (US); Pankaj Nigam, Mason, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/210,345

(22) Filed: Aug. 24, 2005

(51) Int. Cl. A61F 13/15

(52) U.S. Cl. 604/392; 604/385.11

(57) ABSTRACT

A simple disposable absorbent article, which can be a pull-on garment, includes a chassis and an absorbent assembly. A belt is fastenable to the chassis, and both the belt and the chassis can be independently extensible or substantially inextensible. The belt can be fastened to the chassis at opposing ends, at least one of which being releasable and refastenable to induce a tension in the belt and a contractive force on the underlying chassis, thereby enhancing the fit of the garment at the wearer's waist. The belt can further define a frangible region that can be broken to define opposing outer ends, at least one of which can be drawn and refastened to induce the contractive force on the underlying chassis.

### FIELD OF THE INVENTION

The present invention relates to disposable absorbent articles such as disposable diapers and other articles intended for use on incontinent persons and, in particular, relates to an apparatus that enhances the fit of such disposable absorbent articles on the body of the wearer.

### BACKGROUND OF THE INVENTION

Disposable absorbent articles are designed to absorb and contain bodily waste in order to prevent soiling of the body and clothing of the wearer, as well as bedding or other objects with which the wearer comes into contact. Disposable pant-like garments, especially those of the “pull-on” type, include a pair of side edges, each defining corresponding closed side interfaces that, in turn, define an encircled waist opening and a pair of encircled leg openings. Accordingly, pull-on garments can be more easily applied to a wearer, especially a standing wearer, than taped diapers which require manual fastening to define the waist and leg openings and secure the diaper on the wearer.

As the usage of disposable absorbent articles has expanded, the incorporation of additional absorbent article features has correspondingly increased to enhance the performance and appearance of the articles. Unfortunately, the resulting increase in absorbent article complexity has caused the costs of the materials and the manufacturing processes to increase which, in turn, has caused the prices at which these articles are sold to have risen to levels that many potential purchasers around the world cannot afford to pay.

Accordingly, simple disposable absorbent articles can have an extensible chassis that can extend as the articles are applied to the wearer's waist region. Specifically, as such an article is applied to the wearer, portions of the chassis can extend elastically up to the point at which plastic deformation occurs. As a result, a portion of the chassis at the wearer's waist region extended into the region of plastic deformation may fit more loosely about the wearer relative to other portions of the chassis that were not extended to the point of plastic deformation during application of the pant to the wearer.

Furthermore conventional disposable pull-on garments having performance and appearance-enhancing features that lack the extensibility to fit over the hips of the wearer may be designed such that they are slightly oversized relative to the hips and waist of the wearer such that they may be applied to the wearer. Such garments result in a relatively loose fit about the wearer's waist region.

What is therefore needed is an inexpensive apparatus that enhances the fit of an absorbent article about the waist region of the wearer.

### SUMMARY OF THE INVENTION

The present invention provides a belt designed for absorbent articles that can be tightened and fastened to apply a contractive force to the underlying chassis.

In accordance with one aspect of the invention, a disposable pull-on garment includes a chassis, an absorbent assembly attached to the chassis, and a belt member. The chassis defines a front waist region, a back waist region, a crotch region disposed between the waist regions, a waist opening, and a pair of leg openings. The belt member is attached to the chassis in a waist region, and extends across at least a portion of the chassis. The belt member comprises a frangible joint that is breakable to define first and second free ends. The free ends are fastenable to at least one of each other and the chassis to impart a contractive force onto the chassis.

In accordance with another aspect of the invention, a method is provided for securing a pull-on garment at the waist region of a wearer. The garment includes a chassis defining a front waist region, a back waist region, and a crotch region disposed between the waist regions. The front and back waist regions are closed to define a waist opening. The diaper further includes a belt member attached to the chassis, the belt member comprising a frangible joint. The method includes the steps of A) pulling the absorbent article up to the waist region of the wearer such that the waist region is surrounded by the waist opening; B) breaking the frangible joint to define first and second free ends; C) after step (B), attaching at least one of the free ends to one of the chassis and a remaining portion of the belt member, thereby creating a contractive belt force to the chassis.

In accordance with yet another aspect of the invention, a disposable pull-on garment includes a chassis, an absorbent assembly attached to the chassis, and a belt member. The chassis defines a front waist region, a back waist region, a crotch region disposed between the waist regions, a waist opening and a pair of leg openings. The belt member is attached to the chassis at a first location, and extends across at least a portion of the chassis. The belt member defines opposing outer ends attached to the chassis at corresponding attachment zones. At least one of the attachment zones is releasably attached to the chassis, wherein the releasable attachment zone can be released from the chassis and reattached to the chassis in a second location, different from the first location, such that belt applies a contractive belt force to the chassis.

### BRIEF DESCRIPTION OF THE DRAWINGS

In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

FIG. 1 is a perspective view of a disposable pull-on garment constructed in accordance with one aspect of the present invention;

FIG. 2 is a front elevation view of an exemplary pull-on garment having been pulled over the lower torso region of the wearer illustrating portions of the garment that are not securely fit to the wearer's body;

FIG. 3A is a perspective view of an absorbent article similar to FIG. 1 but incorporating a belt aligned with the front waist region in accordance with one aspect of the invention;

FIG. 3B is a perspective view of an absorbent article similar to FIG. 3A but with the belt aligned with the back waist region in accordance with another aspect of the invention;

FIG. 3C is a perspective view of an absorbent article similar to FIGS. 3A-B but with the belt disposed substantially at the side of the article;

FIG. 4 is a side elevation view of a portion of an absorbent article illustrating a belt being tightened to enhance the fit of the article about the wearer;

FIG. 5A is a front elevation view of a portion of a pull-on garment including a belt having a frangible region;

FIG. 5B is a front elevation view of a portion of a pull-on garment as illustrated in FIG. 5A after the frangible region has been broken to define a pair of outer ends;

FIG. 5C is a front elevation view of a portion of a pull-on garment as illustrated in FIG. 5B after the outer ends have been fastened in accordance with one embodiment;

FIG. 5D is a front elevation view of a portion of a pull-on garment as illustrated in FIG. 5B after the outer ends have been fastened in accordance with another embodiment;

FIG. 5E is a front elevation view of a portion of a pull-on garment as illustrated in FIG. 5B after the outer ends have been fastened in accordance with still another embodiment;

FIG. 5F is a front elevation view of a portion of a pull-on garment as illustrated in FIG. 5B after the outer ends have been fastened in accordance with still another embodiment;

FIG. 6 is a perspective view of an absorbent article including a circumferential belt that surrounds both the front and back waist regions;

FIG. 7A is a front elevation view of a portion of an absorbent article as illustrated in FIG. 5A including a belt having a frangible region constructed in accordance with an alternative embodiment;

FIG. 7B is a perspective view of the absorbent article illustrated in FIG. 7A with the frangible region configured in accordance with one aspect of the invention;

FIG. 7C is a perspective view of the absorbent article similar to FIG. 7B but with the frangible region configured in accordance with another aspect of the invention;

FIG. 7D is a front elevation view of a portion of an absorbent article as illustrated in FIG. 7A with the frangible region constructed in accordance with an alternative embodiment;

FIG. 8 is a plan view of a disposable absorbent article prior to being configured as a pull-on garment, with the interior portion of the garment that faces inwardly toward the wearer and contacts the wearer shown facing the viewer, in which the garment is shown in its flat, uncontracted state (i.e., without the contraction induced by elastic members);

FIG. 9 is a plan view of the absorbent article illustrated in FIG. 8 in its flat, uncontracted state, with the exterior portion of the garment that faces outwardly away from the wearer shown facing the viewer;

FIG. 10 is a section view of the garment illustrated in FIG. 8 taken along line 10-10;

FIG. 11 is a section view of the garment illustrated in FIG. 8 taken along line 11-11;

FIG. 12 is a section view of the garment illustrated in FIG. 8 taken along line 12-12;

FIG. 13 is a section view of the garment illustrated in FIG. 8 taken along line 13-13;

FIG. 14A is a schematic perspective view of the garment illustrated in FIG. 8 configured as a pull-on garment showing the side interfaces constructed in accordance with one embodiment of the present invention;

FIG. 14B is a schematic perspective view of the garment illustrated in FIG. 8 configured as a pull-on garment showing the side interfaces constructed in accordance with an alternative embodiment;

FIG. 14C is a schematic perspective view of the garment illustrated in FIG. 8 configured as a pull-on garment showing the side interfaces constructed in accordance with an alternative embodiment;

FIG. 14D is a schematic perspective view of the garment illustrated similar to FIG. 14D showing a belt being attached integrally into one of the side interfaces;

FIG. 15 is a plan view of a pull-on garment constructed in accordance with an alternative embodiment, with the interior portion of the garment that faces inwardly toward the wearer and contacts the wearer shown facing the viewer, shown in its flat, uncontracted state (i.e., without the contraction induced by elastic members) before side flaps are formed by folding portions of the chassis laterally inward;

FIG. 16 is a plan view of the garment illustrated in FIG. 15 in its flat, uncontracted state, with the exterior portion of the garment that faces outwardly away from the wearer shown facing the viewer;

FIG. 17 is a section view of the garment illustrated in FIG. 15 taken along line 17-17;

FIG. 18 is a section view of the garment illustrated in FIG. 15 taken along line 18-18;

FIG. 19 is a section view of the garment illustrated in FIG. 15 taken along line 19-19;

FIG. 20 is a section view of the garment illustrated in FIG. 15 taken along line 20-20;

FIG. 21 is a perspective view of a pull-on garment, with the interior portion of the garment that faces inwardly toward the wearer and contacts the wearer shown facing upward, in which the garment is shown in its contracted state prior to being configured into a pull-on garment (i.e., with the contraction induced by elastic members);

FIG. 22 is a plan view of an exemplary fragment of a formed web material;

FIG. 23 is a plan view of an absorbent assembly, with the interior portion of the absorbent assembly that faces inwardly toward the wearer and contacts the wearer shown facing the viewer, in which the absorbent assembly is shown separate from a chassis to which it is attached in an exemplary garment;

FIG. 24 is a section view of the absorbent assembly illustrated in FIG. 23 taken along line 24-24;

FIG. 25 is a section view of the absorbent assembly illustrated in FIG. 23 taken along line 25-25;

FIG. 26 is a plan view of an exemplary garment having portions removed to illustrate the garment in a stretched configuration.

FIG. 27 is a plan view of an exemplary absorbent article having a fastening device, wherein the absorbent article is positioned in its flat out uncontracted state (i.e., without elastic induced contraction), with the body-facing surface facing the viewer, having portions cut-away; and

FIG. 28 is a plan view of an exemplary fastening device in its fastened configuration.

### DETAILED DESCRIPTION OF THE INVENTION

Definitions

In this description, the following terms have the following meanings:

The term “absorbent article” refers to a device that absorbs and contains liquid and, more specifically, refers to a device that is placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments and the like.

The term “diaper” refers to an absorbent article that is generally worn by infants and incontinent persons about the lower torso so as to encircle the waist and the legs of the wearer and that is specifically adapted to receive and contain urinary and fecal waste. The term “closed side interface” refers to a given side edge (or region adjacent the side edge), wherein a portion of the side edge (or region adjacent the side edge) in the front waist region is joined to a portion of the same side edge (or region adjacent the side edge) in the rear waist region to define closed, encircled leg openings and a closed waist opening. The side interface can be closed with a refastenable or permanent closure member.

The term “pant” (also referred to as “training pant”, “closed diaper”, and “pull-on garment”) refers to disposable garments having a continuous perimeter waist opening and continuous perimeter leg openings designed for infant or adult wearers. A pant can be configured with a continuous or closed waist opening and at least one continuous, closed, leg opening prior to the article being applied the wearer for use. A pant can be preformed by any suitable technique including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened, front waist fastened, rear waist fastened). Examples of suitable pants are disclosed in U.S. Pat. No. 5,246,433; U.S. Pat. No. 5,569,234; U.S. Pat. No. 6,120,487; U.S. Pat. No. 6,120,489; U.S. Pat. No. 4,940,464; U.S. Pat. No. 5,092,861; U.S. Pat. No. 5,897,545; U.S. Pat. No. 5,957,908; and U.S. Patent Publication No. 2003/0233082 A1.

The term “closure member” refers to an element that maintains the article waist and leg openings in a closed, continuous, configuration until the closure member is released. Suitable closure members include a seam, an adhesive, a cohesive, a heat bond, a pressure bond or weld, a tab-and-slot configuration, a hook-and-loop configuration, and the like.

The term “refastenable closure member” refers to a closure member that can be opened and subsequently re-closed, reliably, without destroying the closure member or surrounding diaper components. Examples of refastenable closure members include tabs-and-slots, hooks-and-loops, peelable adhesives, cohesives, and the like

The term “permanent closure member” refers to a closure member that cannot be opened without causing the closure member to fail (i.e., the closure member cannot again be reliably closed). At times, when attempting to open a permanent closure member, surrounding absorbent article component(s) may be damaged or torn. Examples of permanent closure members include adhesives, heat bonds, pressure welds, cohesives, and the like, and further include seams.

The term “seam” refers to an elongated line of junction that attaches two regions of a diaper chassis. Seams can be created via thermal bonds, pressure bonds, ultrasonic bonds, permanent adhesive bonds, permanent cohesive bonds, welds, and stitching. A seam can be configured as a permanent closure member.

The term “cohesive” refers to the property of a material that sticks to itself but does not to any significant degree stick to other materials.

The term “disposable” refers to the nature of absorbent articles that generally are not intended to be laundered or otherwise restored or reused as an absorbent article, i.e., they are intended to be discarded after a single use and, preferably, to be recycled, composted or otherwise disposed of in an environmentally compatible manner.

The term “extensible” refers to any material which, upon application of a biasing force less than 500 grams/inch, is elongatable at least about 20 percent without experiencing catastrophic failure.

The term “longitudinal” refers to a direction running generally parallel to the maximum linear dimension of an element. Directions within ±45° of the longitudinal direction are considered to be “longitudinal”.

The term “lateral” refers to a direction running generally at a right angle to the longitudinal direction. Directions within ±45° of the lateral direction are considered to be “lateral”.

The term “disposed” refers to an element or region being attached and/or positioned in a particular place or position in a unitary structure with other elements.

The term “attached” refers to elements being connected or united by fastening, adhering, bonding, etc. by any method suitable for the elements being attached together and their constituent materials. Many suitable methods for attaching elements together are well-known, including adhesive bonding, pressure bonding, thermal bonding, mechanical fastening, etc. Such attachment methods can be used to attach elements together over a particular area either continuously or intermittently.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition can be permeable to water vapor, i.e., can be “vapor-permeable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables And Nonwovens Association).

The terms “proximal” and “distal”, unless otherwise specified, refer respectively to the location of an element of a structure near to or far from a central axis of the structure, e.g., the proximal edge of a longitudinally extending element of a structure is located nearer to the longitudinal axis of the structure than the distal edge of the same element is located relative to the same longitudinal axis.

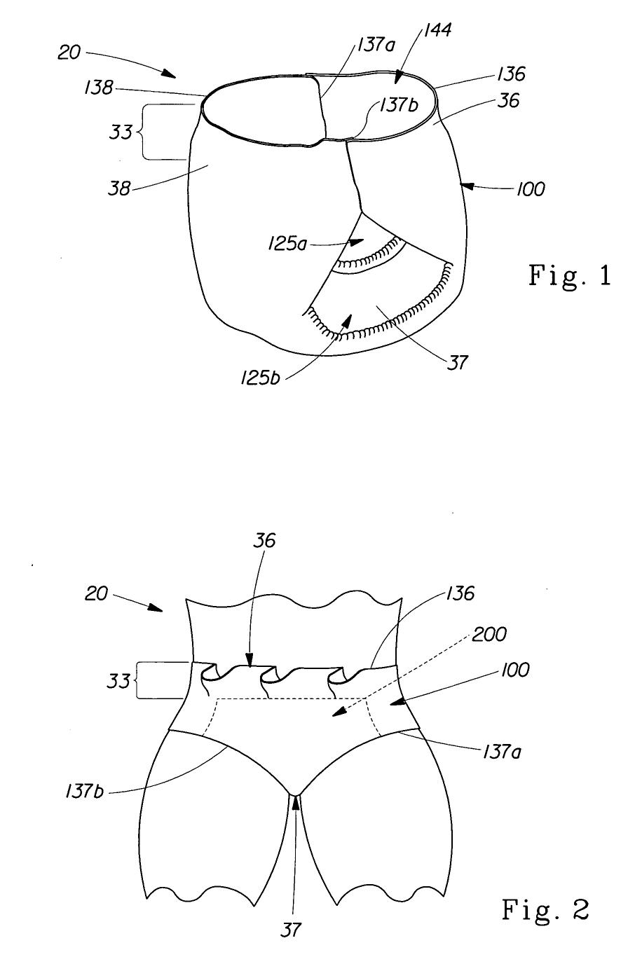

Referring now to FIG. 1, an absorbent article, which can be a training pant, an incontinence pad, a diaper, or other bodily wrap, is illustrated as a pull-on garment 20 including a chassis 100. The chassis 100 includes a front waist region 36, a back waist region 38, and a crotch region 37 disposed between the waist regions. The waist regions 36 and 38 generally comprise those portions of the garment 20 which, when worn, encircle the waist of the wearer. The crotch region 37 is that portion of the garment 20 which, when the garment 20 is worn, is generally positioned between the legs of the wearer.

The chassis 100 defines a left side edge 137a and an opposing right side edge 137b. A portion of the left side edge 137a (or region adjacent the left side edge 137a) at the front waist region 36 is pre-fastened to a portion of the left side edge 137a (or region adjacent the left side edge 137a) at the back waist region 38 to define a closed left side interface 119a that, in turn, defines an encircled left leg opening 125a. Likewise, a portion of the right side edge 137b (or region adjacent the right side edge 137b) at the front waist region 36 is pre-fastened to a portion of the right side edge 137b (or region adjacent the right side edge 137b) at the back waist region 38 to define a closed right side interface 119b which, in turn, defines an encircled right leg opening 125b. The side edges 137a-b, when fastened in the manner described above, combine to form an encircled waist opening 144.

The chassis 100 further defines a front waist edge 136 in the front waist region 36 and an opposing back waist edge 138 in the back waist region 38. When the front waist region and back waist region portions of the side edges 137a-b are fastened, a portion of the chassis waist regions 36 and 38 defines a circumferential belt region 33 that is disposed at or adjacent at least one of the front and back waist edges 136 and 138. The circumferential belt region 33 can abut the waist edges 136 and 138, or it can be spaced below the waist edges. Generally, when the garment 20 is applied to the wearer, the belt region 33 will be generally aligned with the wearer's lower torso region (e.g., the upper waist region). It should be appreciated, however, that the belt region 33 can be disposed anywhere on the garment 20 that could benefit from fit enhancement of the type described in more detail below.

Referring now to FIG. 2, the present invention recognizes that the chassis 100 can be “extensible” which, as used herein, refers to the ability to extend or stretch in a given direction in an amount greater than 20% of its original dimension (including both elastic and plastic deformation) when an extension force of 500 gm/inch is applied to the chassis in the direction of extensibility without undergoing failure. For instance, when the chassis 100 is said to be laterally extensible, the chassis 100 is capable of extending in the lateral direction in an amount greater than 20% of its original lateral dimension. It should be further appreciated that the term “extensible” further encompasses a chassis having an extensibility within a range whose lower end is defined by and between 20%, 25%, and 30%, and whose upper end is defined by and between 40%, 60%, 80%, and 100% with respect to its original dimension along a given direction. It should be furher appreciated that a portion of the extension may result in elastic (i.e., returnable) deformation, while additional extension, that beyond the yield point of the extensible material results in plastic (i.e. permanent) deformation.

Alternatively, the chassis 100 can be configured substantially non-extensible, it being appreciated that most materials exhibit at least a nominal degree of extensibility. Accordingly, the term “substantially non-extensible” as used herein refers to a diaper whose chassis cannot achieve an extensibility (including both elastic and plastic deformation) of greater than 20% of its original dimension when subjected to a force of 500 grams in the direction of the applied force without undergoing failure. For instance, when the chassis 100 is said to be substantially laterally non-extensible, the chassis 100 is incapable of extending in the lateral direction more than 20% its original lateral dimension without ripping, tearing, or the like. It should be further appreciated that the term “substantially non-extensible” further encompasses extensibility no greater than 15%, alternatively 10%, and alternatively still 5% its original dimension. Methods for producing both non-extensible and substantially extensible chassis are described in more detail below.

If the chassis 100 is laterally extensible, the chassis 100 will extend as the article is pulled up to the wearer's waist region. For instance, as the pull-on garment 20 is applied to the wearer, the chassis 100 can extend elastically up to the point at which plastic deformation occurs. The present inventors envision that a portion of the chassis 100 can elastically recover and exert both frictional forces and normal forces against the wearer's waist region up to the point of plastic deformation. While the present inventors believe that the elastic recovery of the extensible chassis can provide adequate fit about the wearer's waist region it may not be sufficient for the purposes of preventing substantial sagging during use, especially during active wear times. The present invention recognizes that the overall fit of the garment 20 can be enhanced, particularly at the wearer's waist region. For instance, a portion of the chassis 100 that has been elastically or plastically deformed during application can fit loosely about the waist region of the wearer, which typically has a circumference less than that of the wearer's hip region. The fit of the garment can be enhanced by a fit enhancement member described hereinafter.

If the chassis 100 is substantially non-extensible, then the chassis 100 should be configured such that the waist opening 144 should have a circumference large enough to pass over the wearer's hips during application, which generally will cause the circumference of the waist opening 144 to be greater than the circumference of the wearer's waist region that typically engages the chassis 100 at the belt region 33. The present inventors recognize that such a pull-on chassis 100 is not likely to achieve a fit at the wearer's waist region that is sufficiently secure for the purposes of preventing substantial sagging during use. The present invention therefore recognizes that the overall fit of the garment 20 can be enhanced, particularly at the wearer's waist region, by inclusion of a fit enhancement member as will now be described.

Description of Belts for Fit Enhancement

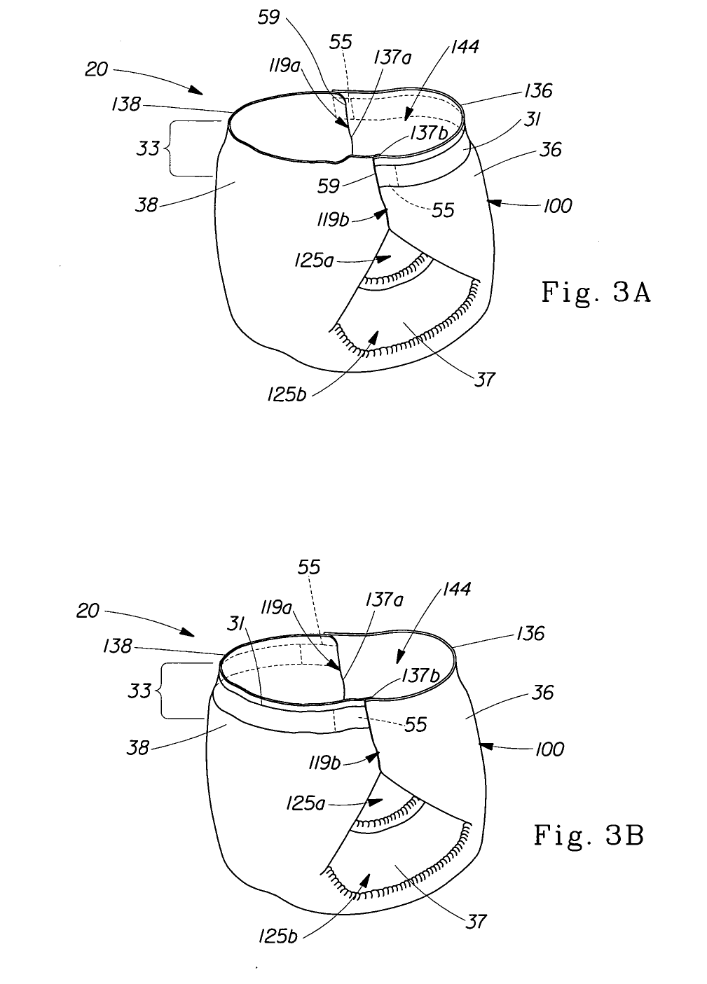

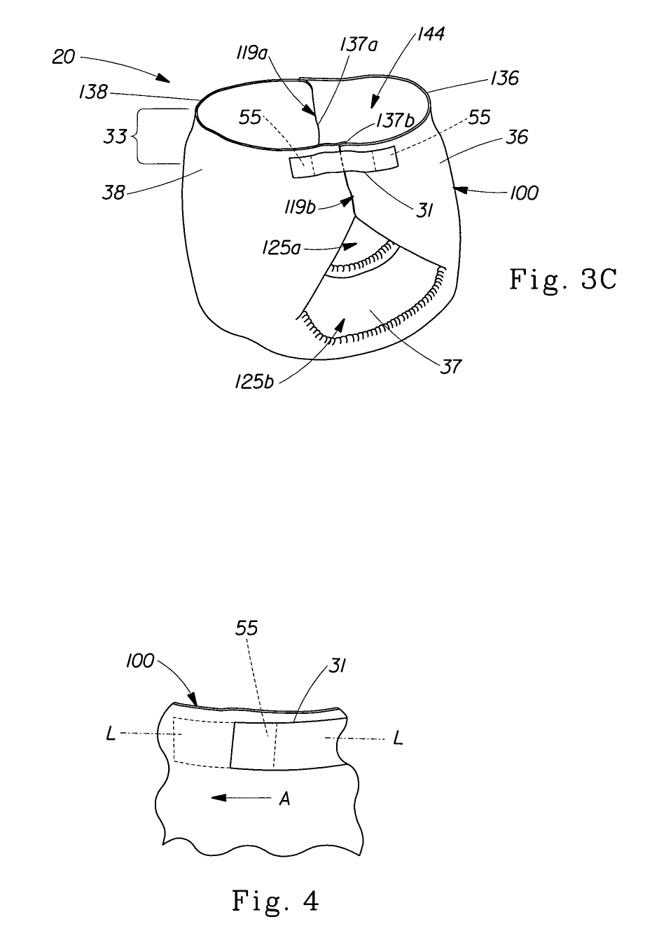

Accordingly, referring to FIGS. 3A-C, certain aspects of the present invention recognize that the pull-on garment 20 can include a fit enhancement member, illustrated as an elongated belt member 31, that is configured to provide a contractive force to the belt region 33 of the chassis 100 (and hence to the wearer's waist region). The belt 31 extends along a longitudinal belt axis L-L (see FIG. 4) and can be configured to be substantially non-extensible (e.g., formed from a non-extensible web material such as a nonwoven, a film, a nonwoven and film laminate, or the like) along the longitudinal belt axis L-L, or alternatively can be configured to be extensible along the direction of the longitudinal belt axis L-L.

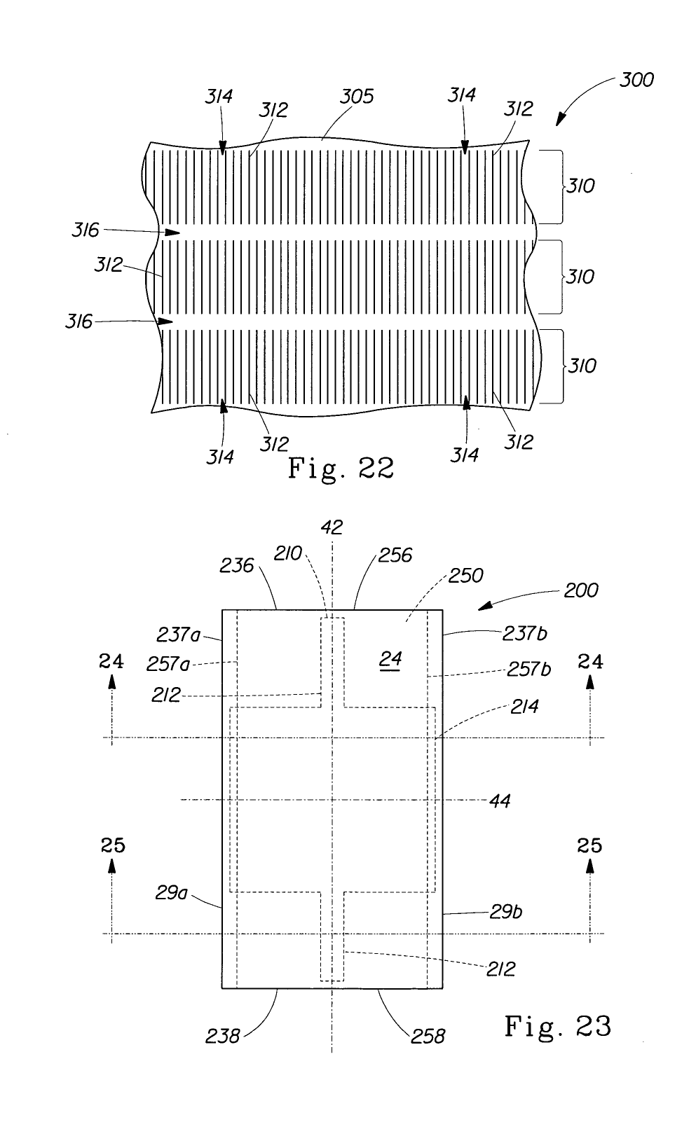

To render the belt 31 extensible, the belt 31 can be formed from a web material that is preformed with longitudinally extending regions in which the original material has been altered by embossing or another method of deformation to create a pattern of generally laterally oriented alternating ridges and valleys, as described in U.S. Pat. No. 5,518,801 (issued May 21, 1996 to Chappell et al), U.S. Pat. No. 5,691,035 (issued Nov. 25, 1997 to Chappell et al), U.S. Pat. No. 5,723,087 (issued Mar. 3, 1998 to Chappell et al), U.S. Pat. No. 5,891,544 (issued Apr. 6, 1999 to Chappell et al), and U.S. Pat. No. 5,968,029 (issued Jan. 19, 1999 to Chappell et al). The formed web material also includes longitudinally extending unaltered regions located between the laterally oriented and longitudinally extending altered regions. When the web is subsequently subjected to an applied elongation along the longitudinal belt axis, the web material exhibits an elastic-like behavior as it extends in the direction of applied elongation and returns to its substantially untensioned condition once the applied elongation is removed, unless the web material is extended beyond the point of yielding, i.e. the point of plastic deformation. This process is described in more detail below with reference to FIG. 22. The web extensibility is adjustable by varying the percentage of the web surface which is comprised of the ridges and valleys.

If the belt 31 (or portions of the belt 31) is extensible, the belt 31 (or the extensible portions) can be either pre-tensioned prior to attachment to the chassis 100 or non pre-tensioned. If the belt 31 is pre-tensioned, the belt 31 will apply a contractive force to the chassis 100 that tends to reduce the circumference of the waist opening 144. If the belt 31 is non pre-tensioned, the belt 31 will not apply a contractive force until the belt region 33 (or other underlying chassis structure) is pulled over a structure (e.g., the wearer's hip region) that extends the chassis 100 and belt 31.

Alternatively, as described in more detail below, at least a portion (or substantially all) of the belt 31 can be ring-rolled and thus rendered extensible as described in U.S. Pat. No. 5,366,782 (issued Nov. 22, 1994 to Curro, et al).

As illustrated in FIG. 3A, the belt 31 includes attachment zones 55 disposed at the opposing distal ends 59 of the belt 31 and adjacent the side edge 137 of the chassis 100. The attachment zones 55 can comprise a permanent attachment such as a heat seal, pressure weld, adhesive, cohesive, or other suitable permanent fastening material. Alternatively, the attachment zones 55 can comprise an adhesive, a cohesive, or mechanical fastener such as hooks or loops that mate with corresponding loops or hooks on the chassis 100 to provide an attachment zone that is releasably (and refastenably) attached to the chassis 100. While the attachment zones 55 are preferably disposed at the distal ends of the belt 31, it should be appreciated that the zones 55 could alternatively be disposed anywhere along the belt 31 that is inward of the distal ends such that the belt 31 can be reliably fastened to the garment 20. The belt 31 could further include additional attachment zones disposed between the outer attachment zones 55 if desired.

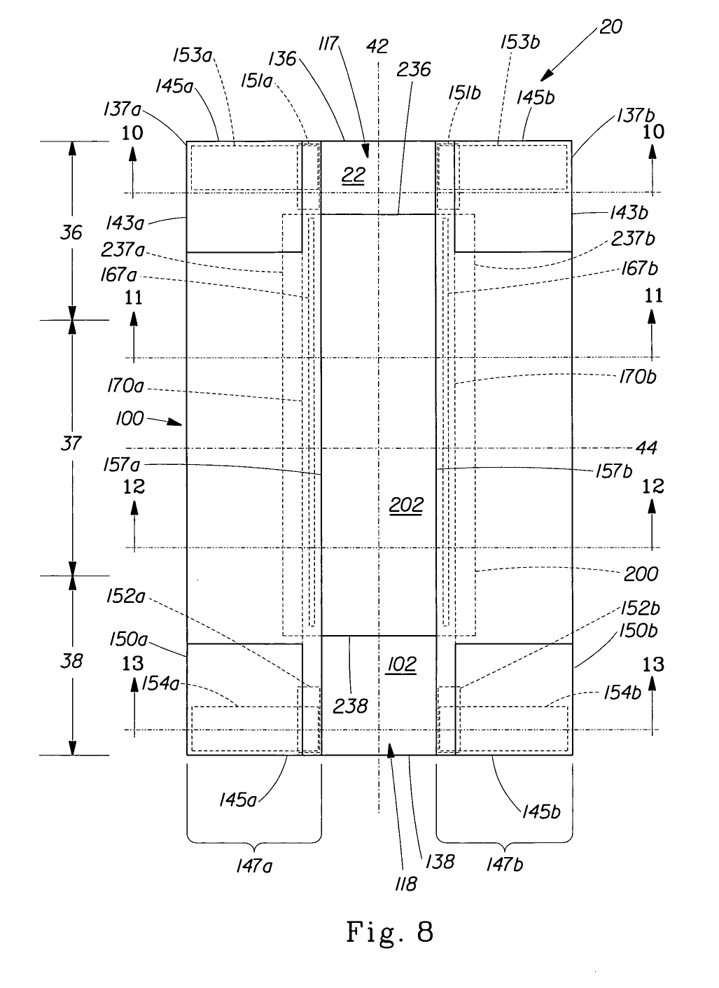

Referring to FIG. 3A, the belt 31 extends laterally across the front waist region 36, and the attachment zones 55 are fastened to the chassis 100 at a location adjacent, and in front of, the closed side interfaces 119a-b. Alternatively, one or both of the attachment zones 55 could be fastened to the chassis 100 at a location further in front of the closed side interfaces 119a-b, i.e. closer to the longitudinal chassis axis 42 (see FIG. 8) than illustrated in FIG. 3A such that the belt 31 extends laterally across only a portion of the width of the front waist region 36. Alternatively still, the attachment zones 55 could be fastened to the chassis 100 behind the closed side interfaces 119a-b such that the belt 31 extends from one side of the back waist region 38 laterally across the front waist region 36 and to the opposing side of the back waist region 38. Alternatively still, one of the attachment zones 55 could be disposed behind one of the side interfaces 119a-b while the other attachment zone 55 could be disposed in front of the opposing side interface such that the belt 31 extends laterally across a portion of the front waist region 36 and a portion of the back waist region 38.

Referring to FIG. 3B, the attachment zones 55 can be fastened to the chassis 100 at a location adjacent, and behind, the closed side interfaces 119a-b such that the belt 31 extends laterally across the back waist region 38. Alternatively, one or both of the attachment zones 55 could be fastened to the chassis 100 at a location farther behind the closed side interfaces 119a-b (i.e., closer to the longitudinal chassis axis 42) than illustrated in FIG. 3B such that the belt 31 extends laterally across only a portion of the back waist region 38. Alternatively, one or both the attachment zones 55 could be fastened to the chassis 100 in front of the closed side interfaces 119a-b such that the belt 31 extends laterally across the back waist region 38 and a portion of the front waist region 36.

Referring now to FIG. 3C, the belt 31 can alternatively provide one attachment zone 55 disposed adjacent, and behind, one of the side interfaces, such as the right side interface 119b as illustrated, while the other attachment zone 55 is disposed adjacent, and in front of, the same side interface 119b such that the belt 31 is disposed substantially at the side of the wearer and in an overlapping relationship with the side interface. Alternatively, or additionally, a second belt can be provided overlapping the opposing side interface having one attachment zone fastened to the chassis 100 at a location adjacent, and behind, the left side interface 119a while the other attachment zone is fastened to the chassis 100 at a location adjacent, and in front of, the same side interface 119a. The attachment zones may also be disposed laterally inward from the side interfaces, i.e. closer to the longitudinal centerline of the chassis, in either the front waist region, back waist region or both front and back waist regions.

Referring now to FIG. 4, the belt 31 can be tightened to enhance the fit of the garment 20 applied to a wearer. Specifically, once the pull-on garment 20 has been applied to the wearer, the user can manually release one of the attachment zone(s) 55 from the chassis 100, apply a tension inducing force to the belt 31 (i.e., by pulling one belt end in a direction away from the opposing belt end along the direction of Arrow A), and re-fastening the released attachment zone 55 to the chassis 100. It should be appreciated that reattachment of the released attachment zone 55 can draw additional chassis material under the belt 31 such that the belt 31 can overlap a greater portion of the chassis periphery compared to the portion of chassis periphery overlapped by the belt prior to releasing the attachment member 55. The resulting tension induced in the belt 31 causes the belt 31 to apply a fit enhancing contractive force to the chassis 100.

It should be appreciated that one attachment zone 55 can be permanently attached to the chassis 100 and that the other attachment zone 55 can be releasably (and refastenably) attached to the chassis 100. Visible indicia can be provided that indicates to the user which attachment zone is releasably and refastenably attached to the chassis 100 and, hence, can be adjusted. Alternatively, both attachment zones 55 can be releasably and refastenably attached to the chassis and adjustable. In certain embodiments comprising a belt wherein both outwardly disposed attachment zones are releasable and refastenable the belt may further comprise attachment zones disposed intermediate the outwardly disposed attachment zones.

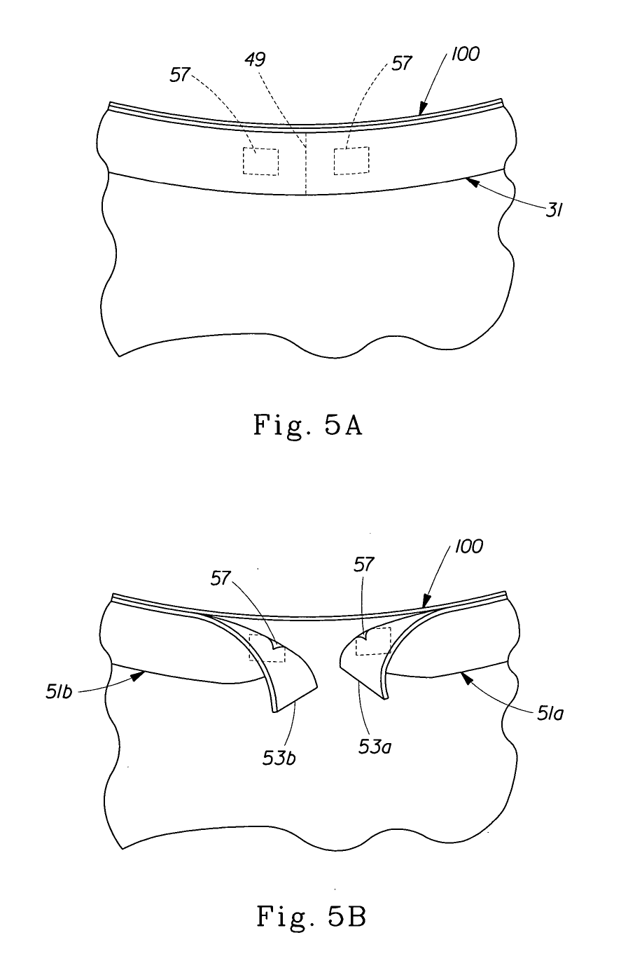

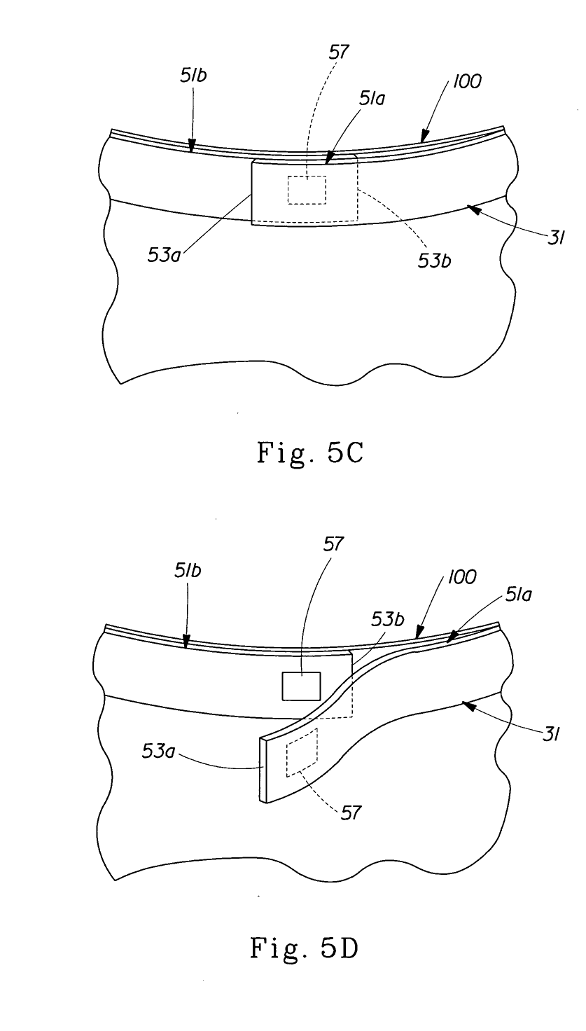

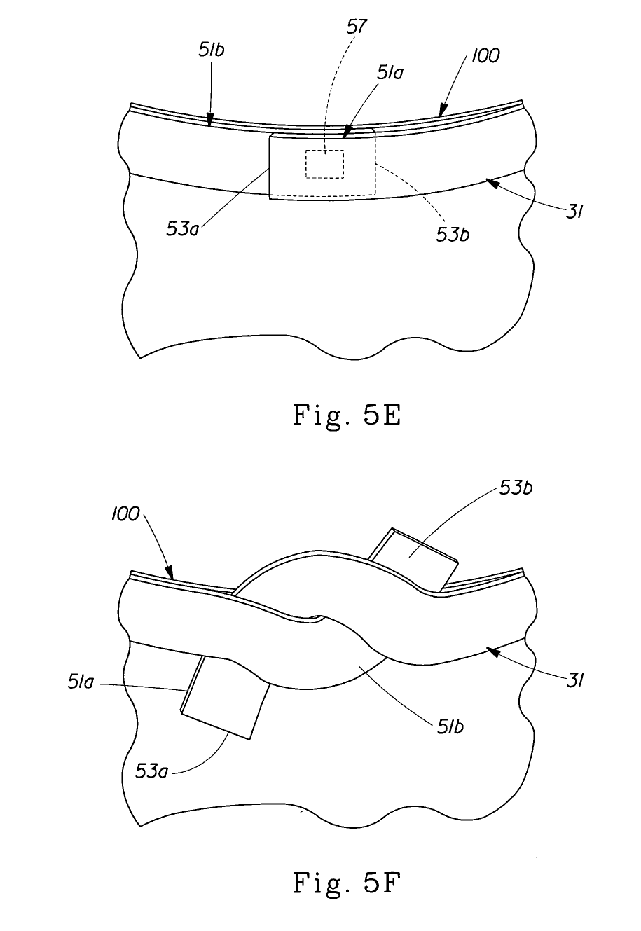

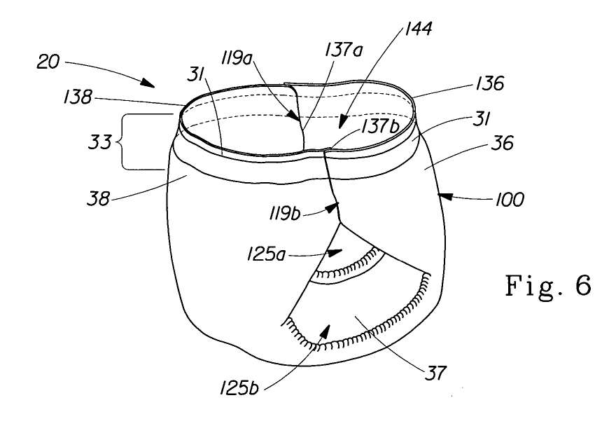

Referring now to FIGS. 5A, the belt 31 constructed in accordance with any of the embodiments illustrated and described above with respect to FIGS. 3A-C can include a frangible joint 49 disposed between two attachment zones 55 positioned in any location described above. The frangible joint 49 is defined as an area or region that is weaker than the surrounding belt regions such that the frangible joint 49 provides a natural tear path when an appropriate force is applied to the belt 31. For instance, the frangible joint 49 can comprise a perforation that can be easily and reliably torn manually by the wearer or caregiver as desired. Referring also to FIG. 6, the present invention appreciates that the frangible joint 49 can be included in a belt 31 that defines a circumferential member surrounding the entire periphery of the chassis 100. The belt 31 can be secured to the chassis 100 via frictional and contractive belt forces, or the belt 31 can be permanently or removably attached to the chassis 100 via any suitable attachment members of the type described above.

It should be appreciated that the frangible joint 49 can be disposed substantially equidistant from the side edges 137a and 137b in either the front or back waist region (or in both regions if two belts 31 are used, or if one circumferential belt is used having two frangible joints). Alternatively, the frangible joint 49 can be disposed closer to one side edge than the other, or the frangible joint 49 can be disposed adjacent one of the side edges 137a and 137b.

At least one fastening zone 57 can be disposed adjacent the frangible joint 49, and can be disposed on the inner (i.e., body-facing) surface of the belt 31 and/or the outer (i.e., garment-facing) surface of the belt 31. As illustrated in FIG. 5A, the fastening zones 57 are disposed on either side of the frangible joint 49 and can comprise any shape or size as desired. The fastening zones 57 can comprise an adhesive, a cohesive, hooks or loops configured or a slot to attach to corresponding loops or hooks, a button, corresponding cohesive surface or any alternative permanent or releasable (and re-fastenable) fastener. If the fastening zones comprise an adhesive or cohesive, a peelable backing can be provided to cover the adhesive or cohesive prior to use as is known by those having ordinary skill in the art. The fastening zones 57 may be of any shape or size to enable adjustment of the belt for proper fit. The fastening zones 57 may cover only a portion of one or both or the belt surfaces or alternatively may cover the full extent of one or both of the belt surfaces.

Referring to FIG. 5B, when the frangible joint 49 is torn, the belt 31 defines a pair of opposing free ends 53a and 53b disposed proximally relative to the attachment zones 55. By “free end”, as used herein, the present invention refers to the ends 53a and 53b being separate or free from each other. Opposing ends of a continuous belt would also be considered to be free ends. The free ends 53a and 53b may or may not be free from the chassis, as is described in more detail below. If the belt 31 is configured as illustrated in FIG. 6, then only one belt segment defines free outer ends 53a and 53b. If the belt 31 is configured as illustrated in FIGS. 3A-3C, then breaking the frangible joint 49 separates the belt 31 into first and second belt segments 51a and 51b, each of which defining a corresponding free outer end 53a and 53b. Because the frangible joint 49 illustrated in FIG. 5A extends substantially vertically, or perpendicular to the longitudinal belt axis L-L (shown in FIG. 4), the outer edges of free ends 53a and 53b also extend substantially vertically and parallel to each other (when the segment(s) 51a-b extend along the longitudinal belt axis L-L).

Referring now to FIGS. 5C-E, once the frangible joint 49 has been broken, the belt 31 can be tightened and refastened to apply a contractive force to the underlying chassis 100 at the belt region 33. For instance, as illustrated in FIG. 5C, the free ends 53a and 53b can be drawn together until they overlap each other, and subsequently fastened via the fastening zone(s) 57 in an interior-to-exterior surface connection (in this regard, the free ends 53a and 53b are said to be attached to each other). Specifically, the fastening zone 57 of one segment can be attached to the fastening zone 57 of the other segment (for instance if the two fastening zones 57 define a hook-and-loop or any alternative connection). Alternatively, the fastening zone 57 of one segment can be attached to the web material of the opposing segment. It should be appreciated that drawing the free ends 53a and 53b of a non-extensible belt in a direction away from their respective attachment zones 55 introduces tension in the belt 31. Alternatively, referring to FIG. 5D, once the frangible joint 49 has been broken, the belt 31 can be tightened and one or both of the fastening zones 57 of the belt segments 51a and 51b can be fastened to the chassis 100. In order to avoid interference between the two belt segments 51a-b when both are designed to be joined to the chassis 100 and thereby permit independent connection to the chassis 100, the belt segments can be vertically offset such that both fastening zones 57 have clearance and can be attached to the chassis 100.

Alternatively still, referring to FIG. 5E, the belt 31 can be pre-fastened to the chassis 100 at one location adjacent the frangible joint 49. Accordingly, once the joint 49 is broken, only one segment (belt segment 51a as illustrated) is free from the chassis 100 and can be attached to the opposing belt segment 51b as illustrated, or can be attached to the chassis 100 as illustrated in FIG. 5D.

Alternatively still, referring to FIG. 5F, once the joint 49 is broken, the user can elect to tighten the belt 31 and tie the belt segments 51a and 51b in any desired knot. It should be appreciated that the belt segments 51a and 51b can be tied in this manner regardless of whether or not belt 31 includes fastening zones 57.

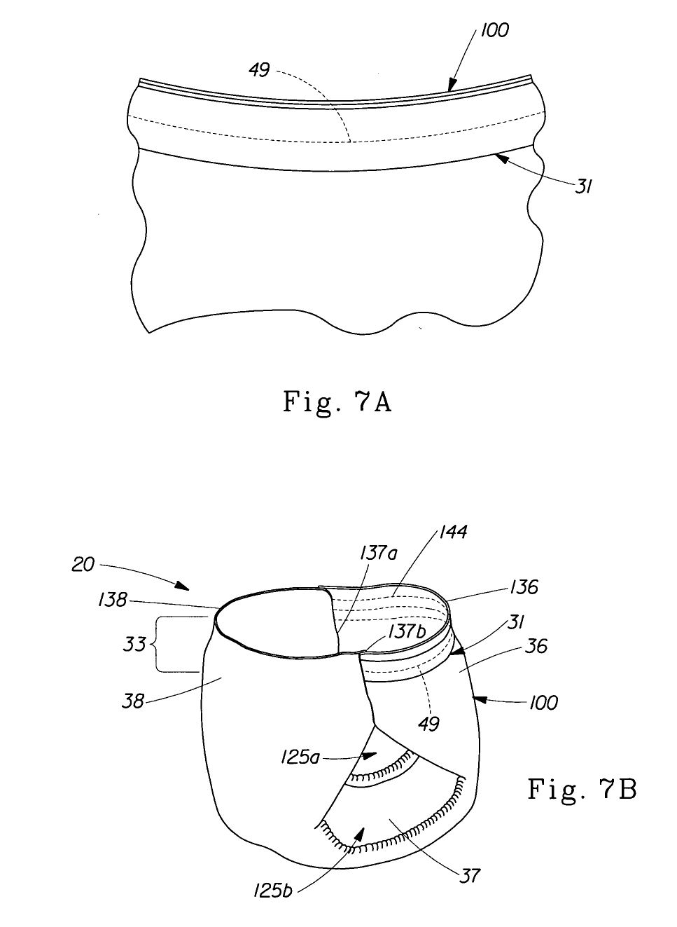

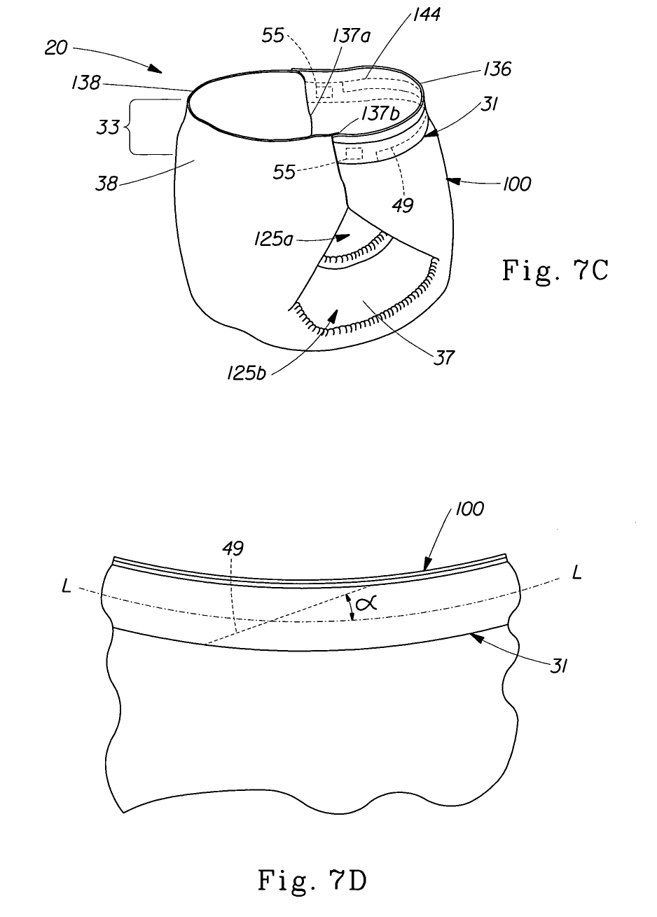

Referring now to FIGS. 7A-D, it should be appreciated that the frangible joint 49 can assume any configuration as desired. For instance, instead of extending substantially perpendicular to longitudinal belt axis L-L as illustrated in FIG. 5A, FIG. 7A illustrates the frangible joint 49 as extending substantially parallel to longitudinal belt axis L-L. As illustrated in FIG. 7B, the frangible joint 49 can span up to the entire distance between opposing side interfaces 119a and 119b. Specifically, the frangible joint 49 extends between, and terminates at or adjacent, the opposing vertical edges of belt 31 or opposing horizontal edges of the belt 31 such that the joint 49 is easily accessible to the user. If, for instance, both opposing ends of the frangible joint 49 extended to the same belt edge, a cutout would be formed when the frangible joint 49 is broken instead of two discrete belt segments. The frangible joint 49 can overlap the attachment zones 55 such that a portion of the attachment zones 55 continue to fasten the belt segments to the chassis, while a portion of the attachment zones 55 is detached and provide fastening zones 57 for the belt segments that are formed after the frangible joint 49 has been broken. As illustrated in FIG. 7C, the frangible joint 49 can terminate inward of one or both side interfaces 119a and 119b. The elongated frangible joint 49 illustrated in FIGS. 7B-C provides elongated belt segments that are particularly configured to be tied together in a knot.

Alternatively still, as illustrated in FIG. 7D, the frangible joint 49 can extend in a direction that defines an angle a between 0 and 90 degrees with respect to the longitudinal belt axis L-L. In one aspect of the invention, the angle α is between 5 and 45 degrees, and alternatively between 5 and 30 degrees. Higher angles formed between the frangible joint 49 and the longitudinal axis L-L provide greater vertical space at the outer ends of the belt segments that can be occupied by fastening zones 57, while lower angles increase the length of the belt segments. For instance, if the resulting belt segments are to be tied in a knot, the angle α can be within the range of 1 and 20 degrees. As described above with respect to FIGS. 6A-C, the frangible joint 49 can span the entire distance between opposing side interfaces 119a and 119b, or the frangible joint 49 can terminate at a position inward of one or both side interfaces.

It should be appreciated that the above examples are only illustrative of exemplary embodiments, and that the frangible joint 49 can assume any geometric configuration as desired to produce corresponding belt segments that can be attached to either each other or the chassis, or tied together in a knot. For instance, while the frangible joint 49 has been illustrated as extending substantially equidistantly between the upper and lower edges of the belt 31, the joint 49 could alternatively be offset towards one of the edges if, for instance, one belt segment was intended to remain secured to the chassis while the other belt segment formed a free end having a sufficient area for a fastening zone 57.

As described above, both the chassis 100 and the belt member 31 can be rendered extensible or substantially non-extensible in the direction of the longitudinal belt axis L-L. The operation of the belt 31 can depend on whether the chassis 100 and/or the belt 31 is extensible, as will now be described with reference to FIGS. 3A-3C, 4, 5A-5F and 6, it being appreciated that the operation of belt 31 is equally applicable to any of the configurations illustrated in FIGS. 7A-D or their alternatives.

If, for instance, the chassis 100 is extensible and the belt 31 is substantially non-extensible, then the belt region 33 can be configured to have a relaxed length, or alternatively an extended length, large enough to pass over the wearer's hips during application. Alternatively, if the belt 31 has a length and position that prevents the chassis 100 from being extended such that it can be pulled up to the wearer's waist, the frangible joint 49 can be broken prior to, or during, application of the garment 20 such that the belt region 33 of the chassis 100 can be extended as it passes over the wearer's hips. Once the garment 20 has been pulled into position at the wearer's waist, the belt 31 can be tightened to create a contractive belt force that is applied to the chassis 100 to enhance the fit of the garment 20 at the wearer's waist when the outer end(s) 53a-b are fastened to the chassis 100 or to each other. It should be appreciated that, because the wearer's waist may have a circumference greater than the belt 31 could accommodate before the frangible joint 49 was broken, the free ends 53a and 53b may be longitudinally spaced from each other even after they are drawn toward each other and attached to the chassis 100. If, on the other hand, the belt 31 defines a length that is sufficient to allow the chassis 100 to be extended in order to pass over the wearer's hips during application of the garment 20 the user need not break the frangible joint 49 until the garment 20 has been applied to the wearer's waist.

As another example, if both the chassis 100 and the belt 31 are extensible, the chassis 100 can define an extended circumference at the belt region 33 that is large enough to fit over the wearer's hips during application. If the belt 31 defines an extended length that is great enough to fit over the wearer's hips, the garment 20 can be applied to the wearer and positioned at the waist before the frangible joint 49 is broken. Once the frangible joint 49 is broken, the belt 31 can then be extended by pulling at least one of the free ends 53a and 53b away from its corresponding attachment zone 55 to provide a compressive belt force that is applied to the chassis. The outer end(s) 53a and 53b can then be fastened as desired. Alternatively, if the belt 31 is less extensible than the chassis 100 such that the chassis 100 cannot be extended to fit over the wearer's hips during application of the garment 20, then the user can break the frangible joint 49 prior to, or during, application of the garment 20. One skilled in the art will recognize that the frangible joint 49 can, if desired, be broken before or during the application of the garment 20 to the wearer even if the belt 31 is of sufficient length or is sufficiently extensible to accommodate the wearer's hips and/or waist region during application of the garment 20. It should be appreciated that the belt 31 can be provided in a pre-tensioned state that provides an initial contractive force to the chassis 100, in which case the chassis 100 will have a contracted waist opening 144.

As yet another example, if both the chassis 100 and the belt 31 are substantially non-extensible, the chassis 100 can be provided having a circumference in the waist region that is at least substantially equal to that of the wearer's hips. If the belt 31 has a sufficient length so as to not restrict the chassis 100 from being pulled over the wearer's hips, the user will have the option of breaking the frangible joint 49 after the garment 20 has been pulled into position at the wearer's waist. Alternatively, if the belt 31 has a length that limits the size of the chassis 100 in the waist region such that the belt prevents the chassis 100 from being pulled over the wearer's hips, the user can break the frangible joint prior to, or during, the application of the garment 20 to the wearer, and subsequently tighten and fasten the free end (s) 53a-b once the garment 20 has been positioned such that the belt 31 is aligned with the wearer's waist.

As still another example, if the chassis 100 is substantially non-extensible and the belt 31 is extensible, the chassis 100 will define a circumference sufficient to allow the chassis to pass over the wearer's hips during application of the garment 20. If the belt 31 is not pre-tensioned, then the garment 20 will fit over the wearer's hips, and the frangible joint 49 can be broken once the garment 20 has been properly positioned at the wearer's waist (or beforehand if desired). Furthermore, the belt 31 can be pre-tensioned and can define an unextended length that is less than that required to enable the belt 31 and chassis 100 to be pulled over the hips of the wearer without extension of the belt 31. Alternatively, or additionally, the pre-tensioned belt 31 may apply a contractive force to the chassis 100 which, in turn, defines a contracted waist opening 144 that cannot be easily extended to fit over the wearer's hips during application of the garment. In this case, the frangible joint 49 should be broken prior to, or during, application of the garment 20. If the belt 31 is sufficiently extensible to allow the belt and chassis to be pulled over the hips of the wearer, the frangible joint 49 can be broken once the garment 20 has been properly positioned at the wearer's waist (or beforehand if desired).

Examples of Exemplary Disposable Pull-On Garments

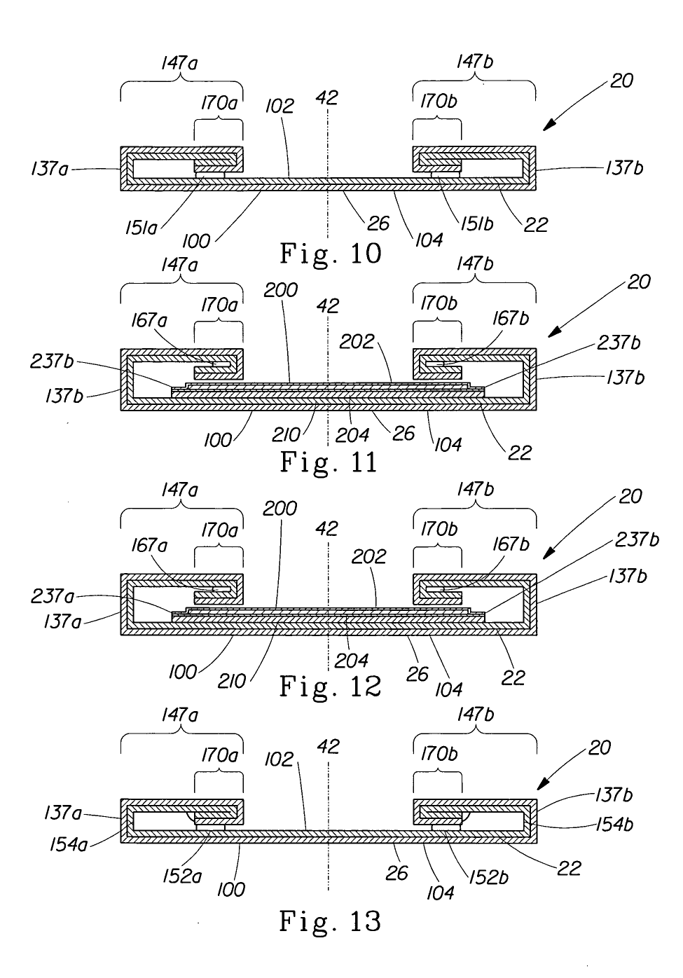

As shown in FIGS. 8-13, one end portion of an absorbent article, illustrated as an exemplary pant-like garment, also referred to as a pant or a pull-on garment 20, is configured as a front waist region 36. The longitudinally opposing end portion of the pull-on garment 20 is configured as a back waist region 38. An intermediate portion of the pull-on garment 20 extending longitudinally between the front waist region 36 and the back waist region 38 is configured as a crotch region 37.

The chassis 100 has a longitudinally extending left side edge 137a and a laterally opposing and longitudinally extending right side edge 137b, both chassis side edges extending longitudinally between the front waist edge 136 and the back waist edge 138. The chassis 100 has an interior surface 102 and an exterior surface 104. The chassis 100 also has a longitudinal chassis axis 42 (extending generally parallel to the lateral axis T of the belt 31) extending equidistantly between side edges 137a and 137b, and a lateral chassis axis 44 extending perpendicular to longitudinal chassis axis 42 (extending generally parallel to the longitudinal belt axis L-L) extending equidistantly between end edges 136 and 138. The longitudinal chassis axis 42 extends through the midpoint of the front waist edge 136 and through the midpoint of the back waist edge 138 of the chassis 100. The lateral chassis axis 44 extends through the midpoint of the left side edge 137a and through the midpoint of the right side edge 137b of the chassis 100. The exemplary chassis 100 shown in FIG. 8 additionally has longitudinally extending and laterally opposing side flaps 147a and 147b that are described in more detail below.

The basic structure of the pull-on garment 20 also includes an absorbent assembly 200 that is attached to the chassis 100. The absorbent assembly 200 has a laterally extending front edge 236 in the front waist region 36 and a longitudinally opposing and laterally extending back edge 238 in the back waist region 38. The absorbent assembly 200 has a longitudinally extending left side edge 237a and a laterally opposing and longitudinally extending right side edge 237b, both absorbent assembly side edges extending longitudinally between the front edge 236 and the back edge 238. The absorbent assembly 200 has an interior surface 202 and an exterior surface 204. The absorbent assembly 200 can be disposed symmetrically with respect to either or both of the longitudinal chassis axis 42 and the lateral chassis axis 44. Alternatively, the absorbent assembly 200 can be disposed asymmetrically with respect to either or both of the longitudinal chassis axis 42 and the lateral chassis axis 44. For example, the absorbent assembly 200 shown in FIG. 8 is disposed symmetrically with respect to the longitudinal chassis axis 42 and asymmetrically with respect to the lateral chassis axis 44. In particular, the absorbent assembly 200 shown in FIG. 8 is disposed asymmetrically toward the front waist region 36.

The respective front edge 236, back edge 238, left side edge 237a, and right side edge 237b of the absorbent assembly 200 can lie inward of the respective front waist edge 136, back waist edge 138, left side edge 137a, and right side edge 137b of the chassis 100, as in the exemplary pull-on garment 20 shown in FIG. 8. Such a configuration in which one or more of the edges of the absorbent assembly 200 lies inward of the corresponding edges of the chassis 100 may be desirable, for example, in order to allow the relatively more flexible layer or layers adjacent to the edges of the chassis to conform to the body of the wearer and thereby form effective gasket-like seals against the skin of the wearer without being constrained by a relatively thicker and relatively less flexible absorbent assembly. Alternatively, one or more of the edges of the absorbent assembly 200 can coincide with the corresponding edge or edges of the chassis 100.

The closed side interfaces 119a-b, in part, define the continuous, closed, left and right leg openings 125a and 125b, respectively, and a continuous, closed, waist opening 144, adapted to fit and gasket the wearer's legs and waist, respectively, as the garment 20 is pulled up to the wearer's lower torso region. The side interfaces 119a-b can be formed into a closed configuration in accordance with any known techniques or methods known in the art. For instance, the interfaces 119a and 119b can be formed with a seam, which may include a bond formed by heat sealing such as ultrasonic bonding, high pressure bonding, RF (radio frequency) bonding, hot air bonding, heated point bonding, and the like as appreciated by one having ordinary skill in the art. Various suitable pant configurations are disclosed in U.S. Pat. No. 5,246,433 (issued on Sep. 21, 1993 to Margaret H. Hasse, et al); U.S. Pat. No. 5,569,234 (issued on Oct. 29, 1996 to Kenneth B. Buell, et al); U.S. Pat. No. 6,120,487 (issued on Sep. 19, 2000 to Gregory Ashton); U.S. Pat. No. 6,120,489 (issued on Sep. 19, 2000 to Larry Johnson, et al); U.S. Pat. No. 4,940,464 (issued on Jul. 10, 1990 to Paul T. Van Gompel); U.S. Pat. No. 5,092,861 (issued on Mar. 3, 1992 to Hironori Nomura et al); U.S. Pat. No. 5,897,545 (issued on Apr. 27, 1999 to Mark James Kline, et al); U.S. Pat. No. 5,957,908 (issued on Sep. 28, 1999 to Mark James Kline, et al); and U.S. Patent Publication No. 2003/0233082 A1 (published on Dec. 18, 2003 to Mark J. Kline, et al).

Alternatively, the closed side interfaces 119a-b can be formed as disclosed in U.S. Pat. No. 5,779,831 (issued on Jul. 14, 1998 to Christoph Schmitz); U.S. Pat. No. 5,772,825 (issued on Jun. 30, 1998 to Christoph Schmitz); U.S. Pat. No. 5,607,537 (issued on Mar. 4, 1997 to Larry Johnson, et al); U.S. Pat. No. 5,622,589 (issued on Apr. 22, 2997 to Larry Johnson, et al); U.S. Pat. No. 5,662,638 (issued on Sep. 2, 1997 to Larry Johnson, et al); U.S. Pat. No. 6,042,673 (issued on Mar. 28, 2000 to Larry Johnson, et al); and U.S. Pat. No. 6,726,792 (issued on Apr. 27, 2004 to Larry Johnson, et al). Various processing methods are well known in the art to provide absorbent pull-on garments. One such process utilizes a final knife followed by a reciprocating tucker blade that pushes the pad from a horizontal orientation to a vertical orientation and a vacuum conveyor belt that holds the pad through a high pressure side seaming unit. The side seaming unit is followed by a slitter that trims the pant edges to provide a finished seam edge. An alternative method disclosed in the aforementioned patents involves cutting the pad in the final knife and bi-folding the pad collecting the pads in a “waterwheel” stacker (a rotary slotted wheel). The bonding is accomplished while the pad is held in place on the rotating wheel.

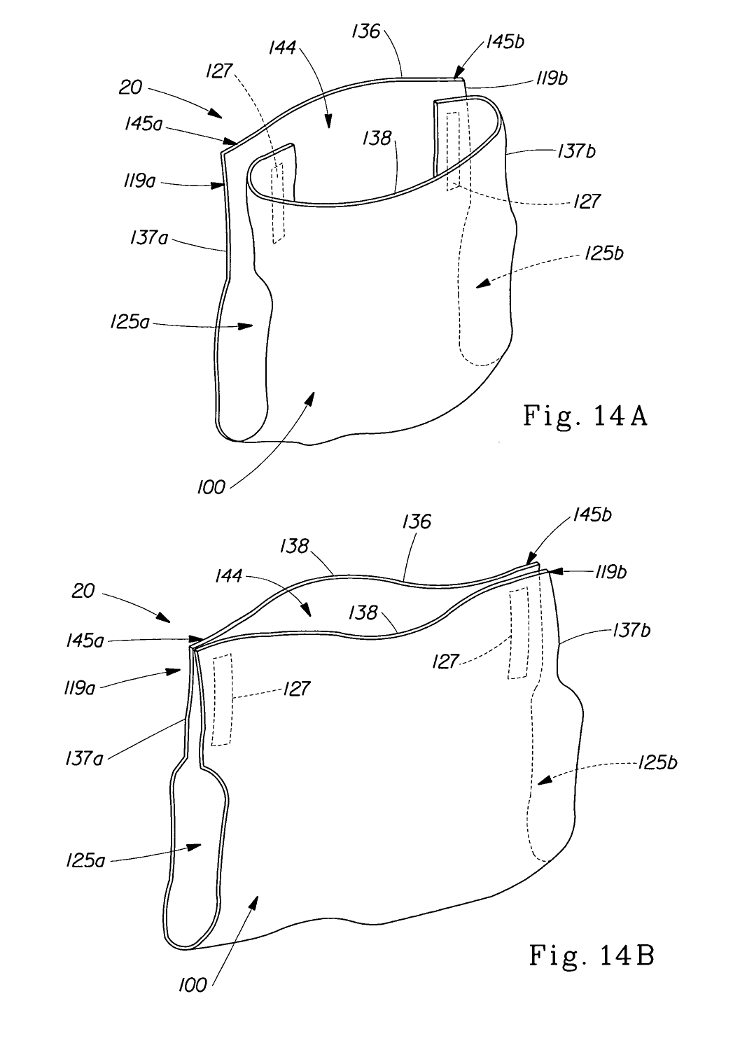

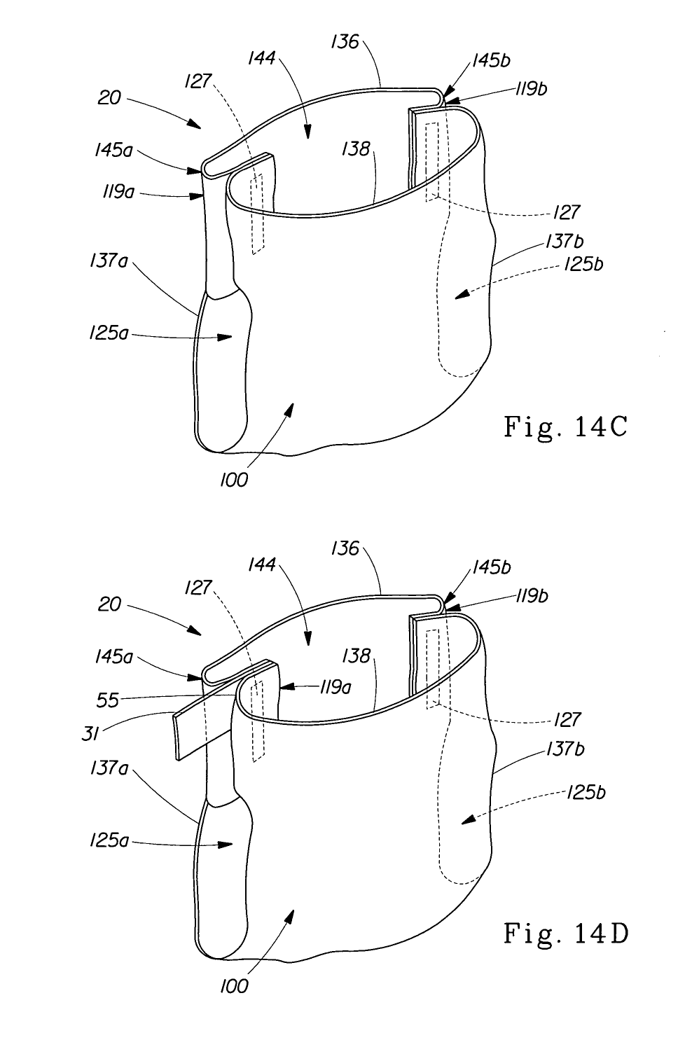

Alternatively, referring to FIGS. 8 and 14A, a left side edge region 145a (defined as a region adjacent the left side edge 137a and including the left side edge 137a) at the front left attachment zone 143a (i.e., in the front waist region 36) can be overlapped with the left side edge region 145a at the back attachment zone 150a (i.e., in the back waist region 38) in an interior surface-to-exterior surface (or vice versa) configuration. Likewise, a right side edge region 145b (defined as a region adjacent the right side edge 137b and including the right side edge 137b) at the front right attachment zone 143b (i.e., in the front waist region 36) can be overlapped with the right side edge region 145b at the back attachment zone 150b (i.e., in the back waist region 38) in an interior surface-to-exterior surface (or vice versa) configuration. Accordingly, the left and right side interfaces 119a and 119b can be closed by attaching the overlapping attachment zones 143 and 150 via any suitable permanent or refastenable closure member 127, such as a seam of the type described above, or an adhesive, a cohesive, a tab-and-slot configuration, or via hook-and-loop attachments. It should be appreciated that joining the side edge regions 145a and 145b causes the side edges 137a and 137b to correspondingly be joined indirectly via the side edge regions 145a and 145b.

Alternatively, referring to FIGS. 8 and 14B, the closed side interfaces 119a and 119b are formed by bi-folding the chassis 100 such that the left and right side edge regions 145a-b, in the front waist region adjacent the front waist edge 136, overlap the left and right side edge regions 145a-b, respectively, in the back waist region adjacent the back waist edge 138 in an interior-to-interior surface configuration. In this configuration, the front waist edge 136 can be substantially aligned with the back waist edge 138 and the side edge 137a in the front and back waist regions can also be substantially aligned as can be the front and back waist regions of side edge 137b. The folded chassis 100 is then attached to the side edge regions 145a-b at the attachment zones 143 and 150, respectively (FIG. 8), using any suitable permanent or refastenable closure member 127, thereby forming a pull-on garment defining continuous left and right leg openings 125a and 125b, respectively, and a continuous, closed, waist opening 144.

Alternatively still, referring to FIGS. 8 and 14C, the closed side interfaces 119a and 119b can be formed by bi-folding chassis 100 such that the left and right side edge regions 145a-b, adjacent the front waist edge 136, overlap the left and right side edge regions 145a-b, respectively, adjacent the back waist edge 138 in an exterior-to-exterior surface configuration. In this configuration, the front end edge 136 can be substantially aligned with the back end edge 138. The folded chassis 100 is then attached at the side edge regions 145a-b at the attachment zones 143 and 150, respectively (FIG. 8), using any suitable permanent or refastenable closure member 127, thereby forming a pull-on garment defining continuous left and right leg openings 125a and 125b, respectively, and a continuous, closed, waist opening 144.

Furthermore, one having ordinary skill in the art will appreciate that the side interfaces 119a-b can be closed via a refastenable closure member that can be nondestructively opened and refastened. Examples of refastenable closure members include hook-and-loop fasteners, snaps, tab-slot fasteners, cohesives, peelable adhesives and the like.

Examples of closure members are described in U.S. Pat. No. 6,432,098 (issued Aug. 13, 2002 to Kline et al); U.S. Pat. No. 6,880,211 (issued Apr. 19, 2005 to Jackson et al); and U.S. Patent Publication No. 2003/0233082 (published Dec. 18, 2003 to Kline et al).

The term “pre-closed” refers to an absorbent article that can be closed by the end user and formed into a pant-like garment prior to applying the garment to the wearer. The term “pre-formed” refers to an absorbent article that has been formed into a pant-like garment in the packaging such that the end user receives the article as a pant-like garment that can be directly applied to the wearer.

Referring now to FIG. 14D, the belt 31 can be attached to the chassis 100 prior to creation of the corresponding side interfaces (illustrated as left side interface 119a. Specifically, the back waist portion of left side edge 137a is attached to the belt 31 at the attachment zone 55, and the attachment zone 55 is in turn attached to the front waist portion of the left side edge 137a such that the front and back waist portions of the left side edge 137a are indirectly attached via the belt 31. Alternatively, or additionally, the front waist portion of the left side edge 137a can be attached to the back waist portion of the left side edge 137a. While FIG. 14D illustrates the belt 31 being integrated into the side interface 119a formed in an exterior-to-exterior surface configuration of the type shown in FIG. 14C, it should be appreciated that the belt 31 could instead be formed in any type of closed interface appreciated by one having ordinary skill in the art.

It should be further appreciated that the belt 31 could be attached to the chassis 100 prior to formation of one or both of the side flaps 147a and 147b and therefore folded inward when the side flaps are formed, or alternatively the belt 31 may be attached to the chassis after formation of one or both of the side flaps whereby the belt can be folded inward in an overlapping relationship with the side flaps. In both of these embodiments, once the chassis is formed into a pull-on pant, the belt can be positioned within one or both of the closed side interfaces 119a-b.

In embodiments in which the belt 31 is in both the front waist region and back waist region, specifically when the belts 31 are disposed at the waist end edges 136 and 138, the belt 31 can be added to the chassis 100 prior to the final knife which is the process step that separates the continuous web of chassis into individual discrete products, thereby severing the belt 31 such that the belt 31 is separated into a front belt portion on one chassis and rear belt portions on a separate previously attached chassis.

Description of the Chassis

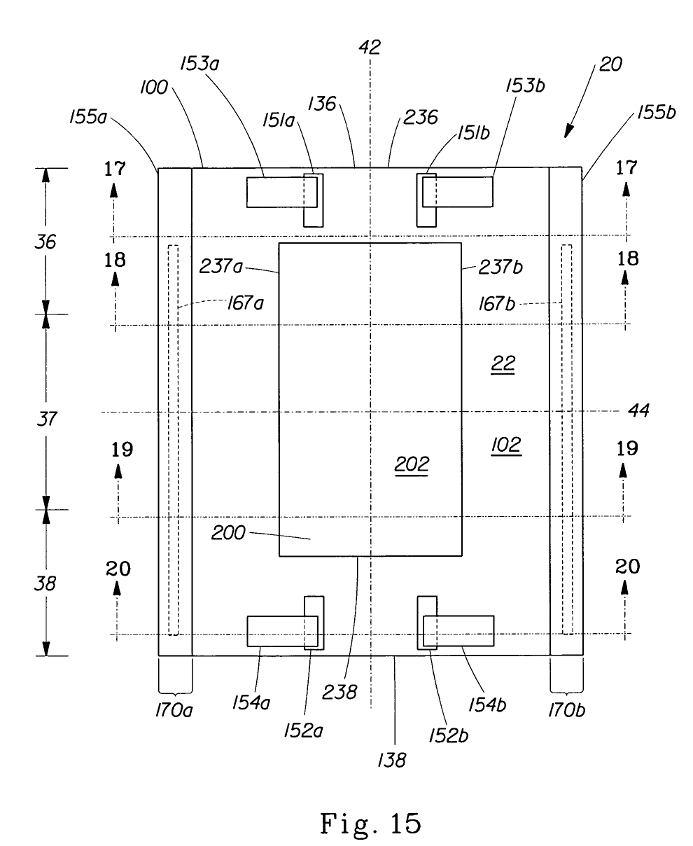

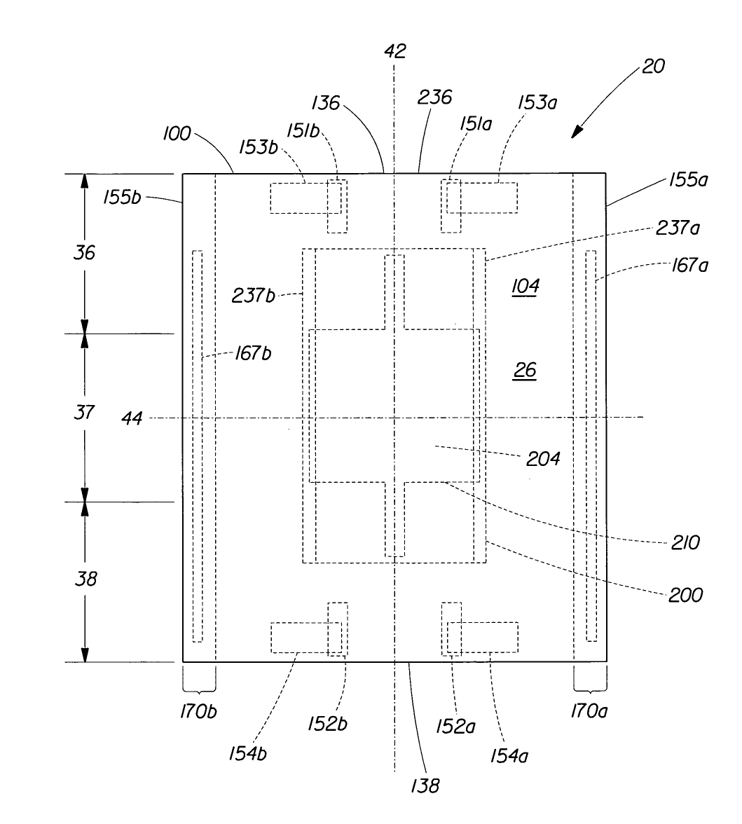

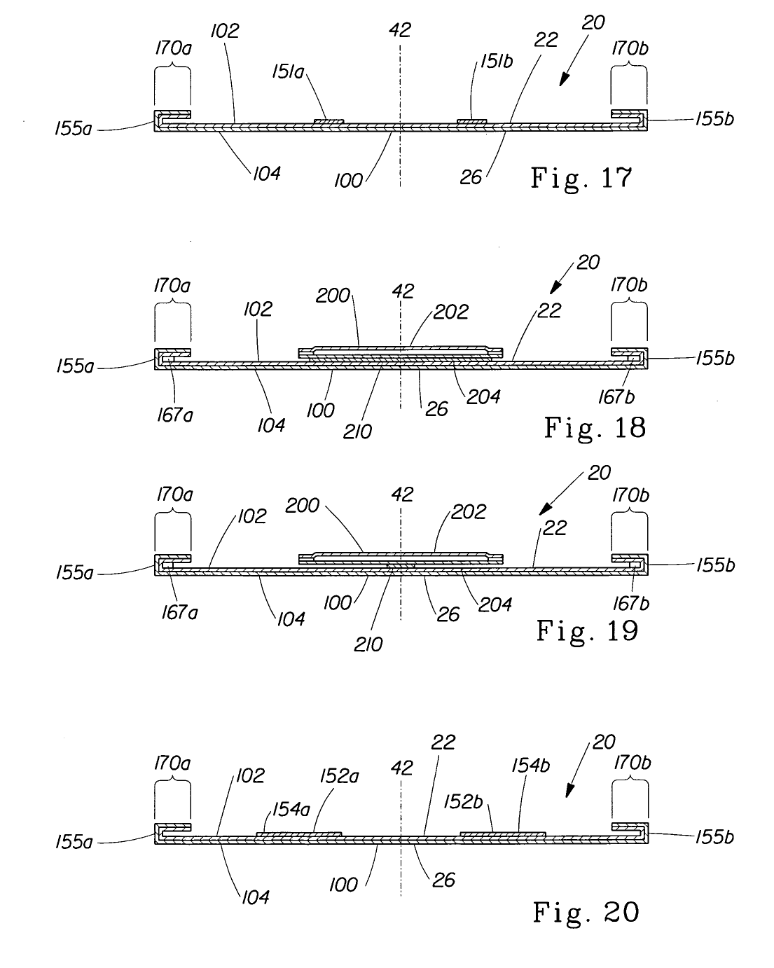

Referring also to FIGS. 15-20, the chassis 100 is shown laid out flat before the side flaps 147a and 147b are formed by folding portions of the chassis 100 laterally inward, i.e., toward the longitudinal chassis axis 42, to form both the respective side flaps 147a and 147b and the side edges 137a and 137b of the chassis 100 as shown in FIGS. 8-13. In this condition of being laid out flat, the chassis 100 has a longitudinally extending left outer side edge 155a and a laterally opposing and longitudinally extending right outer side edge 155b. Both of these chassis outer side edges extend longitudinally between the front waist edge 136 and the back waist edge 138. As is described in more detail below, when the side flaps 147a and 147b are formed by folding portions of the chassis 100 laterally inward, the outer side edges 155a and 155b of the chassis form respective proximal edges 157a and 157b of the side flaps 147a and 147b.

The chassis 100 includes a water-impermeable backsheet 26 defining an exterior surface that is intended to be placed toward clothing that is worn over the pull-on garment 20. The backsheet 26 can be formed from films of polyethylene and other polyolefins, or can alternatively be formed as multi-layer structures, such as laminates of a film and a nonwoven, or alternatively as a dual layer nonwoven laminate as understood by one having ordinary skill in the art. A laminate backsheet can be oriented with the nonwoven disposed exteriorly to provide the feel and appearance of a more cloth-like outermost layer than would be provided by using the film as the outermost layer.

The chassis 100 can further include an inner liner 22 attached to the backsheet 26. As illustrated in FIGS. 15-16, the inner liner 22 can extend to the same width and the same length as the backsheet 26. The inner liner 22 can form a portion of the interior surface 102 of the chassis 100 that is intended to be placed against the body of the wearer. Accordingly, the inner liner 22 can be formed of a soft material that will not irritate the skin of the wearer, and can serve to isolate the skin of the wearer from a portion of the backsheet 26. This may be desired, for instance, when the pull-on garment 20 is worn under conditions in which contact between the skin and a backsheet film could be uncomfortable. Many suitable materials for the inner liner 22 are well known in the art, including rayon and synthetic nonwovens such as spunbonded or carded polypropylene, polyethylene or polyester.

In accordance with an alternative embodiment, one or more of the edges of the inner liner 22 can lie inward of the edges of the backsheet 26. For example, referring to FIG. 8, only the portions of the inner liner 22 lying in the gaps between the front edge 236 of the absorbent assembly 200 and the front waist edge 136 of the chassis 100 and between the back edge 238 of the absorbent assembly 200 and the back waist edge 138 of the chassis 100 are exposed, while the remainder of the inner liner 22 is covered by the absorbent assembly 200 and the side flaps 147a and 147b. Therefore, a laterally extending strip of the inner liner 22 disposed in the gap in the front waist region 36 and a similar laterally extending strip of the inner liner 22 disposed in the gap in the back waist region 38 can suffice to isolate the skin of the wearer from the backsheet 26 in these two gaps.

As shown in FIGS. 8-13, the chassis 100 includes longitudinally extending and laterally opposing side flaps 147a and 147b that are disposed on the interior portion of the garment 20. The side flaps 147a and 147b can be formed by folding portions of the chassis 100 laterally inward to form both the respective side flaps 147a and 147b and the side edges 137a and 137b of the chassis 100. Alternatively, the side flaps 147a and 147b can be formed by attaching an additional layer or layers to the chassis 100 at or adjacent to each of the respective side edges 137a and 137b of the chassis 100.

Portions of a film backsheet 26 that are folded laterally inward to form the side flaps can contact the skin of a wearer during the use of the pull-on garment 20. However, the alternating ridges and valleys in such a film backsheet that has been deformed in order to make it extensible can provide channels through which air can pass to alleviate any concern regarding such contact of the film backsheet with the skin.

In embodiments in which portions of the chassis 100 are folded laterally inward to form the side flaps 147a and 147b, the chassis 100 can simply be folded loosely or can be creased along a portion of each of its side edges 137a and 137b. For example, it may be desirable to form creases along portions of the side edges 137a and 137b in the crotch region 37 in order to impart a more finished appearance to the garment 20. Alternatively or in addition to creasing, a portion of each of the folded side flaps 147a and 147b adjacent to the side edges 137a and 137b can be attached to the interior surface 102 of the chassis 100 to achieve a similar result.

The left side flap 147a defines a proximal edge 157a and the right side flap 147b defines a proximal edge 157b. In the exemplary garment 20 shown in FIG. 8, the proximal edge 157a and the proximal edge 157b lie laterally inward of the respective left side edge 237a and right side edge 237b of the absorbent assembly 200, and the left side flap 147a and the right side flap 147b thus overlap the absorbent assembly 200. Such an overlapped configuration may be desirable in order to impart a more finished appearance to the garment 20 than that imparted by a non-overlapped configuration. Alternatively, the left side flap 147a and the right side flap 147b do not overlap the absorbent assembly 200, for instance when the proximal edge 157a and the proximal edge 157b lies laterally outward of the respective left side edge 237a and right side edge 237b of the absorbent assembly 200.

Referring again to FIG. 8, the left side flap 147a and the right side flap 147b extend the full length of the chassis 100 between the front waist edge 136 and the back waist edge 138. Such a full length configuration may be desirable in order to minimize the amount of waste material and the difficulty associated with the manufacture of the pull-on garment 20, especially when the method used to manufacture the garment 20 requires the introduction of the material or materials for the chassis 100 in the form of a continuous web or multiple continuous webs. Alternatively, the side flaps can be shorter and extend less than the full distance between the front waist edge 136 and the back waist edge 138. Such a shorter configuration may be desirable in order to minimize the total amount of material used in the manufacture of the pull-on garment 20.

Each of the side flaps 147a and 147b is attached to the interior surface 102 of the chassis 100 in attachment zones located in the front waist region 36 and in the back waist region 38. For example, the side flaps 147a and 147b are attached to the interior surface 102 of the chassis 100 in front longitudinally oriented adhesive attachment zones 151 and back longitudinally oriented adhesive attachment zones 152 (more clearly visible in FIG. 15). In particular, the left side flap 147a is attached to the interior surface 102 of the chassis 100 in opposing longitudinally oriented adhesive attachment zones 151a and 152a. Attachment zone 151a is disposed adjacent the proximal edge 157a of left side flap 147a near the front waist edge 136, and attachment zone 152a is disposed adjacent the proximal edge 157a near the back waist edge 138. Similarly, the right side flap 147b is attached to the interior surface 102 of the chassis 100 in opposing longitudinally oriented adhesive attachment zones 151b and 152b. Attachment zone 151b is disposed adjacent the proximal edge 157b of right side flap 147b near the front waist edge 136, and attachment zone 152b is disposed adjacent the proximal edge 157b near the back waist edge 138. The adhesive attachment zones can have equal areas or can be unequal in area. For example, the front longitudinally oriented adhesive attachment zones 151a and 151b can be of one size and the back longitudinally oriented adhesive attachment zones 152a and 152b can be of another size.

Additionally, or alternatively, the side flaps 147a and 147b can be attached to front and back laterally oriented adhesive attachment zones 153 and 154, respectively. Specifically, the left side flap 147a is attached to the interior surface 102 of the chassis 100 in opposing laterally oriented adhesive attachment zone 153a and 154a. Adhesive zone 153a is disposed adjacent the front waist edge 136 and adhesive attachment zone 154a is disposed adjacent the back waist edge 138. Similarly, the right side flap 147b is attached to the interior surface 102 of the chassis 100 in opposing laterally oriented adhesive attachment zones 153b and 154b. Attachment zone 153b is disposed adjacent the front waist edge 136 and attachment zone 154b is disposed adjacent to the back waist edge 138. The adhesive attachment zones can have equal areas or can be unequal in area. For example, the front laterally oriented adhesive attachment zones 153a and 153b can be of one size and the back laterally oriented adhesive attachment zones 154a and 154b can be of another size.

Alternatively, each attachment zone can extend laterally across the full width of the respective side flap. For example, a laterally oriented adhesive attachment zone can extend laterally from the chassis left side edge 137a to the left side flap edge 157a and thereby attach the entire width of the left side flap 147a adjacent to the front waist edge 136 to the interior surface 102 of the chassis 100. In embodiments in which the front edge 236 or the back edge 238 of the absorbent assembly 200 coincides with the respective front waist edge 136 or back waist edge 138 of the chassis 100 and the side flaps 147a and 147b overlap the absorbent assembly 200, the side flaps 147a and 147b can be attached to the absorbent assembly 200 instead of, or in addition to, being attached to the interior surface 102 of the chassis 100.

Between the attachment zones, the proximal edges 157a and 157b of the side flaps 147a and 147b remain unattached to the interior surface 102 of the chassis 100 or to the absorbent assembly 200. Also between the attachment zones, each side flap preferably includes a longitudinally extensible flap elastic member that is attached adjacent to the proximal edge of the side flap by any of many well-known means. Each such flap elastic member can be attached over its entire length or over only a portion of its length. For example, such a flap elastic member can be attached only at or near its longitudinally opposing ends and can be unattached at the middle of its length. Such a flap elastic member can be disposed in the crotch region 37 and can extend into one or both of the front waist region 36 and the back waist region 38. For example, in the exemplary chassis 100 shown in FIG. 8, an elastic strand 167a is attached adjacent to the proximal edge 157a of the left side flap 147a and extends into both the front waist region 36 and the back waist region 38. Similarly, an elastic strand 167b is attached adjacent to the proximal edge 157b of the right side flap 147b and extends into both the front waist region 36 and the back waist region 38.

Each flap elastic member can be enclosed inside a folded hem. For example, in the exemplary chassis 100 shown in FIGS. 11 and 12, the elastic strand 167a is enclosed inside a hem 170a formed adjacent to the proximal edge 157a of the left side flap 147a and the elastic strand 167b is enclosed inside a hem 170b formed adjacent to the proximal edge 157b of the right side flap 147b. Alternatively, the flap elastic member can be sandwiched between two layers of the chassis, e.g., between the layers of a laminate backsheet or between a backsheet and an inner liner. As another alternative, the flap elastic member can be attached on a surface of the chassis 100 and remain exposed.

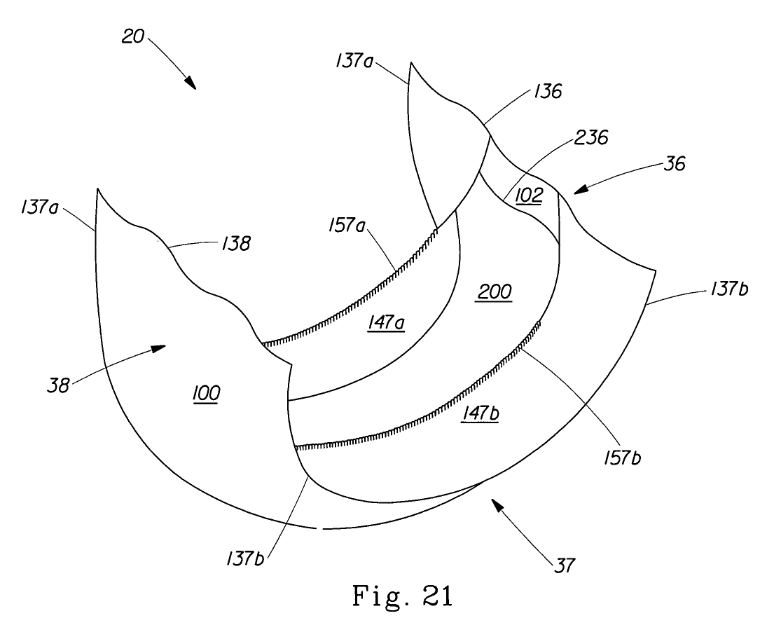

When stretched, the flap elastic member disposed adjacent each side flap edge allows the side flap edge to extend to the flat uncontracted length of the chassis 100 as shown in FIG. 8. When allowed to relax, the flap elastic member contracts to gather the portion of the side flap edge along which the flap elastic member is attached and thereby make the relaxed length of the side flap edge less than the flat uncontracted length of the chassis. For example, when the exemplary garment 20 is in a relaxed condition as shown in FIG. 21, the elastic strand 167a contracts to gather the proximal edge 157a of the left side flap 147a and the elastic strand 167b contracts to gather the proximal edge 157b of the right side flap 147b. The contractive forces of the elastic strands 167a and 167b are transmitted at the respective front attachment zones 151a and 151b to the interior surface 102 of the chassis 100 at the front waist region 36. Similarly, the contractive forces of the elastic strands 167a and 167b are transmitted at the respective back attachment zones 152a and 152b to the interior surface 102 of the chassis 100 at the back waist region 38. These contractive forces pull the front waist region 36 and the back waist region 38 toward each other and thereby bend the garment 20 into a “U” shape in which the interior of the “U” shape is formed by the portions of the garment 20 that are intended to be placed toward the body of the wearer. Because the proximal edge 157a remains free between the attachment zones 151a and 152a, the contractive force of the elastic strand 167a lifts the proximal edge 157a away from the interior surface 102 of the chassis 100. Similarly, because the proximal edge 157b remains free between the attachment zones 151b and 152b, the contractive force of the elastic strand 167b lifts the proximal edge 157b away from the interior surface 102 of the chassis 100. As shown in FIG. 21, this lifting of the proximal edges 157a and 157b when the garment 20 is in the relaxed condition lifts the side flaps 147a and 147b into position to serve as side barriers adjacent to the side edges 237a and 237b of the absorbent assembly 200.

When the garment 20 is worn, the relaxed “U” shape generally conforms to the body of the wearer such that the front waist region 36 and the back waist region 38 encircle the waist and the legs of the wearer. When the pull-on garment 20 is worn in this manner, the elastic strands 167a and 167b tend to hold the lifted proximal edges 157a and 157b of the side flaps 147a and 147b in contact with the body of the wearer and thereby form seals to help prevent the leakage of deposited bodily waste out of the pull-on garment 20. The lateral spacing of the lifted proximal edges 157a and 157b is selected to allow the deposit of bodily wastes from the lower torso of the wearer into the space between the lifted side flaps 147a and 147b and thereby directly onto the absorbent assembly 200. The width of each of the side flaps 147a and 147b in effect becomes its height when the free portion of its proximal edge 157a and 157b, respectively, is lifted and the side flap serves as a side barrier to leakage. This height preferably is selected to allow the lifted proximal edges 157a and 157b to fit into the leg creases of the body of the wearer at the same time as the absorbent assembly 200 is held in contact with the body.

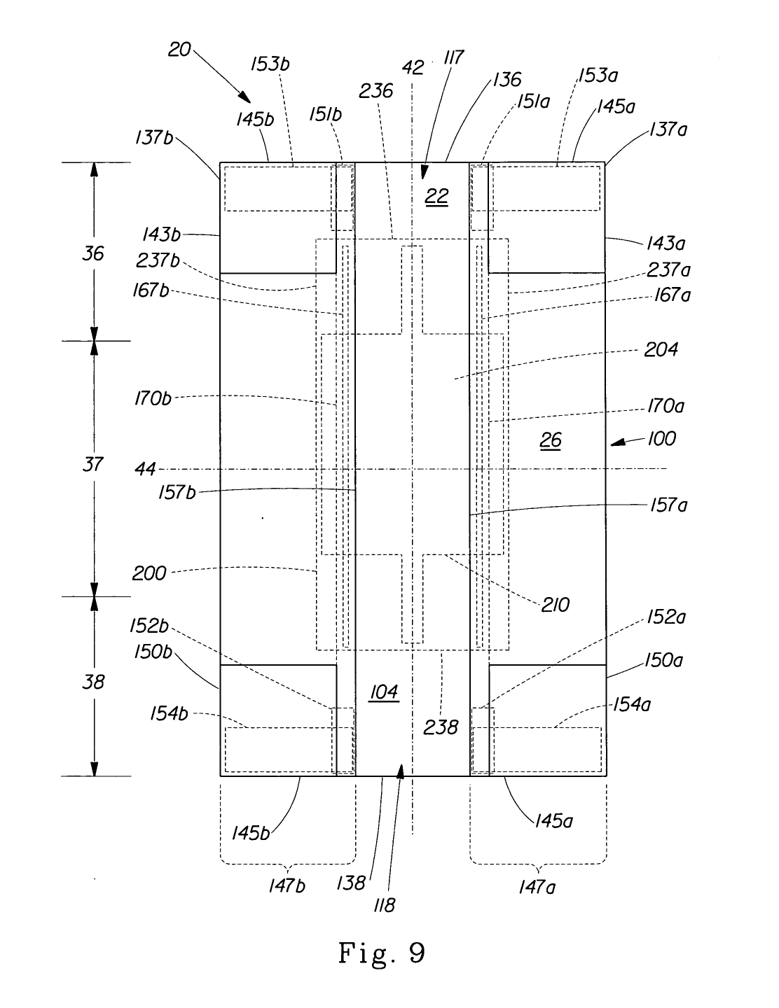

As illustrated in FIGS. 8-9, the chassis 100 can have a generally rectangular shape, which may be desirable in order to minimize the amount of waste material and the difficulty associated with the manufacture of the pull-on garment 20. Alternatively, the chassis side edges 137a and 137b may not be straight, but instead can be curved and/or notched, thereby giving an overall shape in plan view of an hourglass or of an “I” to the pull-on garment 20. Such a non-rectangular configuration may be desirable in order to impart a tailored appearance to the pull-on garment 20 when it is worn, and further to impart an impression that the pull-on garment 20 will fit comfortably between the legs of a wearer. Any one of many well-known techniques can be used to form a non-rectangular configuration of the chassis. For example, the chassis 100 can be made narrower in the crotch region 37 than at the waist edges 136 and 138 by removing laterally distal portions from the chassis 100 to make its lateral dimension at and adjacent to the lateral chassis axis 44 smaller than its lateral dimension at and adjacent to the front waist edge 136 and smaller than its lateral dimension at and adjacent to the back waist edge 138. Alternatively, a portion of each of the side edges 137a and 137b can be folded laterally inward in order to achieve the same result. Such folded portions of the side edges 137a and 137b can be creased or attached, or both creased and attached, in order to prevent their unfoldment.

Part or all of the chassis 100 can be made extensible to a degree greater than the inherent extensibility of the material or materials from which the chassis is made (e.g., the backsheet 26, the inner liner 22, or both). Advantageously, the extensible chassis 100 can exhibit an elastic-like behavior in the direction of elongation without the use of added elastic materials. The elastic-like behavior can be modified and/or provided as desired in a web material 325 (FIG. 22) as described below. The additional extensibility may be desirable in order to allow the chassis 100 to conform to the body of a wearer during movement by the wearer. The additional extensibility may also be desirable, for example, to allow the user of a pull-on garment 20 including a chassis 100 having a particular size before extension to extend the front and/or back waist regions 36 and 38 to enable the pull-on garment to be pulled over the hips of the wearer and then to contract to encircle the waist of an individual wearer whose waist circumference is typically smaller than the circumference as measured at the hips of the wearer. Such extension of the waist region(s) can give the pull-on garment 20 a generally hourglass shape, so long as the crotch region 37 is extended to a relatively lesser degree than the waist region(s), and can impart a tailored appearance to the pull-on garment 20 when it is worn. In addition, the additional extensibility may be desirable in order to minimize the cost of the pull-on garment 20. Specifically, a lesser amount of material is needed in order to make a diaper capable of being properly fit onto a given size of a wearer when the material is made extensible as described.

Additional extensibility in the chassis 100 in the lateral direction is relatively more useful than additional extensibility in the longitudinal direction. The abdomen of the wearer is likely to expand when the wearer changes posture from standing to sitting and the corresponding abdominal expansion increases the circumference that is encircled by the waist edges of the chassis 100, rendering lateral extension of the waist region or regions particularly advantageous.

Additional lateral extensibility in the chassis 100 can be provided in a variety of ways. For example, a material or materials from which the chassis 100 is made can be pleated by any of many known methods. Alternatively, all or a portion of the chassis 100 can be made of a formed elastic-like web material or a formed laminate of web materials like those described in U.S. Pat. No. 5,518,801 (issued May 21, 1996 to Chappell et al), U.S. Pat. No. 5,691,035 (issued Nov. 25, 1997 to Chappell et al), U.S. Pat. No. 5,723,087 (issued Mar. 3, 1998 to Chappell et al), U.S. Pat. No. 5,891,544 (issued Apr. 6, 1999 to Chappell et al), and U.S. Pat. No. 5,968,029 (issued Jan. 19, 1999 to Chappell et al). An exemplary fragment 320 of such a formed web material 325 is shown in FIG. 22. This formed web material 325 includes distinct laterally extending regions 310 in which the original material has been altered by embossing or another method of deformation to create a pattern of generally longitudinally oriented alternating ridges 312 and valleys 314. The formed web material 325 also includes laterally extending unaltered regions 316 located between the laterally extending altered regions 310.

Such a formed web material 325 can be laterally extended beyond its original dimension with the application of relatively less force than that required to extend the same material to the same extent when undeformed. In particular, the effects of an application of opposing divergent forces directed generally perpendicular to the ridges 312 and valleys 314 include an extension of such a formed web material along an axis between the opposing forces and the generation of a resistive contractive force, primarily in the unaltered regions 316. This resistive force is relatively smaller than the resistive force that is generated by the same material in its unaltered form when extended to the same extent, at least up to an extension at which the ridges and valleys in the altered regions flatten and begin to contribute to the resistive force. Thus, such formed web materials exhibit an extensible behavior resembling that of traditional elastic materials in the range of extensibility that is useful for the type of lateral extension desired for use in absorbent articles. However, such formed web materials can be made of relatively less expensive materials that are not inherently elastic and, thus, their use can provide an advantage in terms of the cost of manufacturing the absorbent articles.

The range of extensibility of a web material or a laminate that is formed as described in the Chappell et al. '801 patent can be controlled by the degree of deformation of the altered regions and can be varied from near zero to a maximum that is dependent upon the original material. For example, the materials used in the chassis 100 (e.g., the backsheet 26) of the exemplary pull-on garment 20 can typically be formed to provide any range of extensibility from a minimum of 20% to a maximum of more than 100 percent of the original dimension. In some embodiments of the present invention, a portion of the chassis 100 can have a level of extensibility within a range whose lower end is defined by and between 20%, 25%, and 30%, and whose upper end is defined by and between 40%, 60%, and 80%. The requisite levels of extensibility are achieved by application of an opposing divergent force in the direction of extensibility of preferably less than 1,000 grams/inch, more preferably less than 700 grams/inch. However, it should be easily appreciated that any particular value for the maximum extensibility in the range from approximately twenty percent to approximately 100 percent can be selected to suit a particular choice of the original size of the garment 20 and the range of sizes of the intended wearers. In particular, a diaper having a specific unextended waist opening circumference can be suitable for use on wearers having waist circumferences ranging from equal to this unextended waist opening circumference up to the maximum extensibility.

When the web 325 is subjected to an applied elongation, the web material exhibits an elastic-like behavior as it extends in the direction of applied elongation and returns to its substantially untensioned condition once the applied elongation is removed, unless the web material is extended beyond the point of yielding. The web extensibility is adjustable by varying the percentage of the web surface which is comprised of the ridges 312 and valleys 314. This can be achieved, for instance, by modifying the widths of the ridges 312 and valleys 314, and the spacing between adjacent ridges 312 and valleys 314. A higher percentage of area coverage of the web material 325 by the ridges 312 and valleys will increase the overall extensibility of the web 325. The web 325 is able to undergo multiple cycles of applied elongation up to the yield point without losing its ability to substantially recover. Accordingly, the web 325 is able to return to its substantially untensioned condition once the applied elongation is removed (e.g., as the chassis 100 is pulled over the wearer's waist region during use).

The front laterally central portion 117 and the back laterally central portion 118 of the chassis 100 between the attachment zones 151-154 where the side flaps 147a and 147b are attached to the interior surface 102 of the chassis 100 adjacent to the respective waist edges 137 and 138 can have a different range of extensibility from the portions of the chassis 100 in the attachment zones. Additionally or alternatively, the laterally central portions 117 and 118 can be extensible to a greater or lesser degree when subjected to a given level of opposing tensile forces, and can thus be more easily or less easily extensible than the portions of the chassis in the attachment zones 151-154. For example, if the chassis 100 is made uniformly extensible across its entire width prior to the formation of the side flaps 147a and 147b, the double layering in the areas of the attachment zones after the formation of the side flaps can have an effect of decreasing the degree of lateral extensibility of those areas under a given level of opposing tensile forces, such as by the side flaps acting as parallel “springs” that must be extended in order to extend the underlying attached portion of the chassis. As another example, the altered regions in the laterally central portions of the chassis can be deformed to a greater or a lesser degree than the altered regions in the attachment zones to render the laterally central portions more easily or less easily extensible than the respective portions in the attachment zones. Such differential range of extensibility and/or differential relationship of tensile force to extensibility may be desirable. For example, when the waist regions are laterally extended by a user when applying a pull-on garment to the body of a wearer, each waist region is typically subjected to a generally uniform level of opposing tensile forces across its entire width, so long as the user grasps the garment 20 at or adjacent to the laterally opposing side edges 137a and 137b. If the laterally central portion of the chassis is less easily extensible than the portions in the attachment zones, the lateral spacing between the proximal edges 157a and 157b of the side flaps 147a and 147b will increase less under a given level of applied tensile forces than if the laterally central portion were equally easily extensible or more easily extensible than the portions in the attachment zones 143a-b and 150a-b. This effect of minimizing the change in the lateral spacing between the side flaps 147a and 147b can help to ensure that the pull-on garment 20 fits as intended on the body of the wearer by, for example, making it more likely that the proximal edges 157a and 157b of the side flaps 147a and 147b will fit into the leg creases of the body while the pull-on garment 20 is being worn.

Any of a variety of extensible materials can be formed as described in the Chappell et al. '801 patent. For example, a film, a nonwoven, or a laminate of either or both of these materials can be formed to provide the desired extensibility. It is also possible to modify such a material in more than one way while forming it to provide extensibility. For instance, a film that is originally formed to resist the permeation of vapor through its thickness and to contain fine particles of a granular filler material such as calcium carbonate can be treated as described in the Chappell et al. '801 patent to simultaneously provide extensibility and create small holes that allow water vapor to pass through its thickness. Thus, the film can simultaneously be rendered extensible and breathable. Alternatively, a portion of the backsheet 26 can be ring-rolled and thus rendered highly extensible as described in U.S. Pat. No. 5,366,782 (issued Nov. 22, 1994 to Curro, et al). Specifically, a ring-rolling apparatus includes opposing rolls having intermeshing teeth that incrementally stretch and thereby plastically deform the material forming backsheet 26 (or a portion thereof) thereby rendering the backsheet 26 extensible in the ring-rolled regions. In one embodiment, the backsheet 26 can be ring-rolled in a portion of at least one of the front or back waist regions while other regions may comprise a structured elastic-like formed web material. The chassis may be ring-rolled across the entire width in one or both of the waist regions or alternatively may be ring-rolled over only a portion of the chassis width. In yet another embodiment the chassis may be ring-rolled in the portion of the chassis 100 wherein the side flaps 147 overlap and are joined to the chassis 100 in attachment zones 151, 152, 153, and 154.

Furthermore, once the garment 20 has been positioned on the lower torso region of the wearer, the web 325 enables the garment 20 to apply a contractive force at the front and back waist regions 36 and 38, respectively, to the wearer's body at a level greater than 100 grams, alternatively greater than 200 grams, and alternatively still greater than 300 grams. It may also be desired that the chassis applies a contractive force at the waist regions 36 and 38 that is less than 2,000 grams, alternatively less than 1,500 grams and alternatively still less than 1,000 grams. As described in the Chappell et al. '801 patent, the resistive force exerted by the web 325 (i.e., the contractive force) in response to an applied elongation can be modified. Specifically, the web can be designed to yield virtually any resistive force which is less than that of the base web material by adjusting the percentage of the web surface which is comprised of the first and second regions. With respect to the web material, if the material has a higher percentage of area coverage comprising ridges 312 and valleys 314, the resultant resistive force of the exerted by the against an applied elongation for a given material composition and cross-sectional area will be decreased.

Extension versus force and contractive force can be determined by ASTM 882-02 with the following modifications. A sample representative of the extensible material disposed in the waist region should be collected for the test. In the test a 5.08 cm by 15.24 cm (2 inch by 6 inch) sample is cut from the material such that the edges are straight. The sample is clamped into the tensile tester. The clamps are attached 10.16 cm (4 in) from each other on the sample. The sample is pulled steadily at a speed of 2.54 cm/min (1 in/min) to 20% extension and then immediately returned to 0% (4 in. spacing between the clamps) at the same steady speed. Data, extension in mm and force in grams, should be collected at a rate of at least 1 data point per second. The data can be graphed to provide a curve of % extension versus force such that the extension at various tensile/contractive forces can be determined. The extension force can be determined by the extension curve and the contractive force can be determined by the return curve. This test should be repeated at 30, 40, 50, 60, 70, 80, 90 and 100% extension using a new specimen for each test. A representative sampling should be made for each condition.

To compare the extension force and contractive force of one pull-on garment to another, the diaper in question is applied to a representative group of wearers within the specified size range of the diaper and the circumferential waist dimension of the diaper and/or wearer is determined. The circumferential waist dimension of the diaper as worn is then compared to the diaper waist circumference in a new unextended state. The % extension is derived by the following:
(As-worn waist circumference−original waist circumference)/original waist circumference)

Once the percentage waist extension is calculated, a correlating force can be established using the above-described method. It should thus be appreciated that, for a given diaper, a force to %extension relationship can be determined as described above.

Description of the Absorbent Assembly

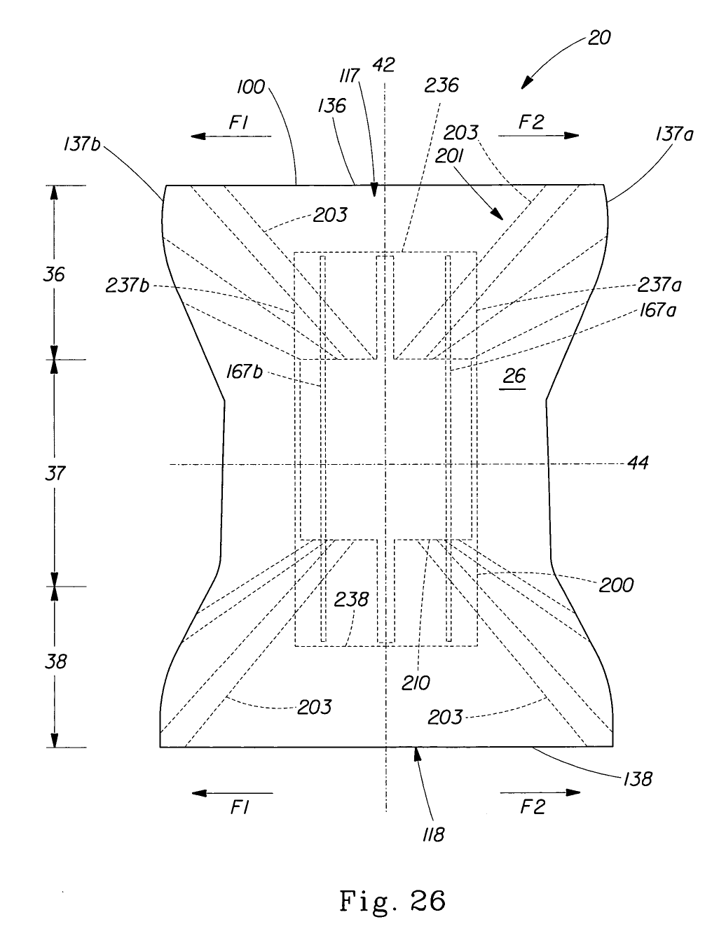

Referring to FIGS. 23-25, the absorbent assembly 200 includes an absorbent core 250. The absorbent core 250 has a laterally extending front edge 256 in the front waist region 36 and a longitudinally opposing and laterally extending back edge 258 in the back waist region 38. The absorbent core 250 also has a longitudinally extending left side edge 257a and a laterally opposing and longitudinally extending right side edge 257b, both absorbent core side edges extending longitudinally between the front edge 256 and the back edge 258. Any or all of the respective front edge 256, back edge 258, left side edge 257a, and right side edge 257b of the absorbent core 250 can lie inward of the respective front edge 236, back edge 238, left side edge 237a, and right side edge 237b of the absorbent assembly 200. For example, in the exemplary absorbent assembly 200 shown in FIG. 23, the absorbent core 250 has its left side edge 257a and right side edge 257b located laterally inward of, respectively, the left side edge 237a and right side edge 237b of the absorbent assembly 200. Alternatively, one or more of the edges of the absorbent core 250 can coincide with the corresponding edge of the absorbent assembly 200. For example, in the exemplary absorbent assembly 200 shown in FIG. 23, the front edge 256 and back edge 258 of the absorbent core 250 coincide with the respective front edge 236 and back edge 238 of the absorbent assembly 200.

The absorbent assembly 200 can be attached to the chassis 100 over any part or the whole of the area of the absorbent assembly 200. In one aspect of the present invention, the absorbent assembly 200 is attached on its exterior surface 204 to the chassis 100, and in particular to the backsheet 26, in a cruciform attachment pattern, i.e., in an attachment pattern that forms or is arranged in a cross or “+” shape. The cruciform attachment pattern can be contiguous, i.e., all of its portions can be touching or connected throughout the pattern in an unbroken sequence. Alternatively, the cruciform attachment pattern can include detached portions and thereby lack contiguity but still be arranged such that the shape of the overall pattern is a cruciform. For example, a discontiguous cruciform attachment pattern can include a longitudinally extending portion disposed along the longitudinal axis and separate left and right laterally distal portions disposed along or adjacent to the lateral axis and thereby form a cruciform as the shape of the overall pattern.

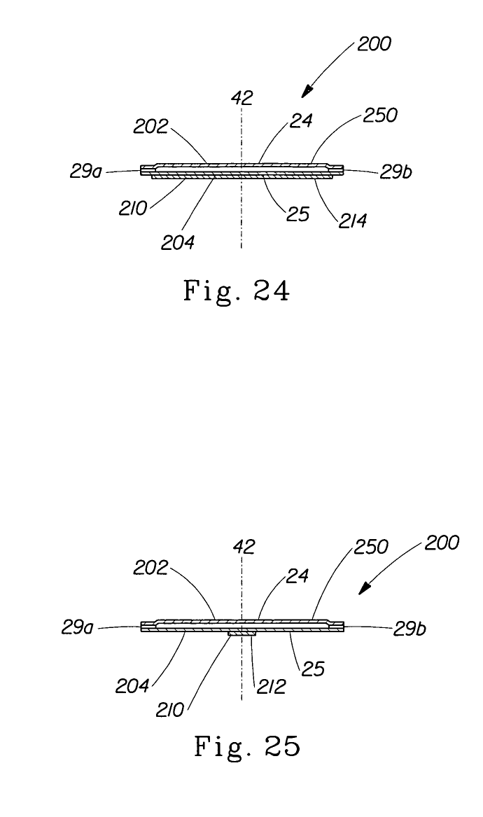

An exemplary contiguous cruciform attachment pattern 210 is shown in FIGS. 20 and 23-25. The portions of the chassis 100 that lie outside such a cruciform attachment pattern are not restrained by attachment to the absorbent assembly 200 and therefore remain extensible. In particular, a relatively narrow longitudinally extending portion 212 of a cruciform attachment pattern 210 like that shown in FIGS. 23-25 leaves the majority of the width of the chassis 100 in the front waist region 36 and in the back waist region 38 freely extensible and thereby allows extension of the chassis 100 in the lateral direction in these regions. A relatively wide laterally extending portion 214 of a cruciform attachment pattern 210 like that shown in FIGS. 23-24 prevents the portion of the chassis 100 in the crotch region 37 to which the absorbent assembly 200 is attached from shifting relative to the absorbent assembly 200 in that region. A relatively wide laterally extending portion 214 of a cruciform attachment pattern 210 can also contribute to the effectiveness of the side flaps 147a and 147b when the elastic strands 167a and 167b lift the proximal edges 157a and 157b into contact with the body of the wearer. For example, if the chassis 100 in the crotch region 37 were free to shift laterally inward, i.e., toward the longitudinal chassis axis 42 such that the left side edge 137a and/or the right side edge 137b moved toward the longitudinal chassis axis 42, the side flaps 147a and 147b might easily distort and fail to maintain contact with the body. However, because the relatively wide laterally extending portion 214 of the cruciform attachment pattern 210 restrains the chassis 100 over a relatively wide portion of the width of the crotch region 37, the side flaps 147a and 147b are better supported at their bases while being lifted by the elastic strands 167a and 167b.

The cruciform attachment pattern 210 in FIGS. 23-25 extends laterally from near the left side edge 237a to near the right side edge 237b of the absorbent assembly 200 at and adjacent to the lateral chassis axis 44, but does not extend laterally to this extent over the full length of the absorbent assembly 200. Similarly, the cruciform attachment pattern 210 in FIGS. 23-25 extends longitudinally from near the front edge 236 to near the back edge 238 of the absorbent assembly 200 at and adjacent to the longitudinal chassis axis 42, but does not extend longitudinally to this extent over the full width of the absorbent assembly 200. Alternatively, the cruciform attachment pattern 210 can extend to any or all of the side edges 237a and 237b and the front edge 236 and the back edge 238 of the absorbent assembly 200. For example, the cruciform attachment pattern 210 can extend laterally from the left side edge 237a to the right side edge 237b of the absorbent assembly 200, but can extend longitudinally only a part of the distance from the front edge 236 to the back edge 238 of the absorbent assembly 200. Within the extent of the cruciform attachment pattern 210, the absorbent assembly 200 can be attached to the chassis 100 continuously or intermittently. For example, a film of an adhesive can be applied continuously over the entire area of the cruciform attachment pattern and then used to continuously attach the absorbent assembly to the chassis. As an alternative example, an adhesive can be applied discontinuously at and inside the boundaries of the cruciform attachment pattern, such as in the form of dots, stripes, beads, spirals, etc., and then used to attach the absorbent assembly to the chassis.

The cruciform attachment pattern 210 can be disposed symmetrically with respect to either or both of the longitudinal chassis axis 42 and the lateral chassis axis 44 of the chassis 100. Alternatively, the cruciform attachment pattern 210 can be disposed asymmetrically with respect to either or both of the longitudinal chassis axis 42 and the lateral chassis axis 44. For example, the cruciform attachment pattern 210 shown in FIG. 23 is disposed symmetrically with respect to the longitudinal chassis axis 42 and asymmetrically with respect to the lateral chassis axis 44. In particular, the cruciform attachment pattern 210 shown in FIG. 23 is disposed asymmetrically toward the front waist region 36. Also, the laterally extending portion 214 of the cruciform attachment pattern 210 can be located distant from the lateral chassis axis 44 and the longitudinally extending portion 212 of the cruciform attachment pattern 210 can similarly be located distant from the longitudinal chassis axis 42. In addition, the cruciform attachment pattern 210 can be disposed symmetrically with respect to either or both of the side edges 237a and 237b and the front edge 236 and the back edge 238 of the absorbent assembly 200. For example, the cruciform attachment pattern 210 shown in FIG. 23 is disposed symmetrically with respect to both the side edges 237a and 237b and the front edge 236 and the back edge 238, i.e., the cruciform attachment pattern 210 shown in FIG. 23 is centered on the absorbent assembly 200. Alternatively, the cruciform attachment pattern 210 can be disposed asymmetrically with respect to either or both of the side edges 237a and 237b and front edge 236 and back edge 238 of the absorbent assembly 200, i.e., the cruciform attachment pattern 210 can be disposed off-center on the absorbent assembly 200.

It should be appreciated that the portion of the chassis 100 that is attached to the absorbent assembly 200 may not be extensible to any significant degree unless an extensible adhesive is used. Advantageously, the cruciform attachment pattern 210 enables attachment of the absorbent assembly 200 to the chassis 100 while, at the same time, providing a significant portion of the chassis 100 that overlaps the absorbent assembly 200 to be free from the chassis 100, particularly in areas in the front waist region 36 and the back waist region 38. Accordingly, the cruciform attachment pattern 210 enables the chassis 100 to be more extensible than an absorbent article whose chassis is connected substantially to a surface of the absorbent assembly or about the periphery of the absorbent assembly. The increased chassis extensibility is useful when, for instance, donning the garment 20 on the wearer.

Referring now to FIGS. 22 and 26, the backsheet 26, by virtue of the ridges 312 and valleys 314 described above, is extensible at regions that are free from the absorbent assembly 200, including regions that are disposed directly beneath the absorbent assembly 200 and free from the absorbent assembly 200. The extensibility of portions of the backsheet 26 increases as the portions become increasingly distant from the laterally extending portion 214 of the cruciform pattern 210.

When the pull-on garment 20 is pulled onto the body of the wearer, a force will be applied by the garment 20 to the waist region of the user to secure the garment 20 onto the body of the wearer. Forces applied to the garment 20 during application are simulated in FIG. 26 as opposing laterally outward forces F1 and F2 applied to the left and right side edges 137a and 137b, respectively, in the front waist region 36 and back waist region 38. Upon application of forces F1 and F2, the backsheet 26 deforms to a significantly greater degree in the waist region than in the crotch region thereby creating angled lines of tension 203 directed from portion 214 of the cruciform pattern 210 to the side edges 137a and 137b in both the front and back waist regions 36 and 38. These angled lines of tension 203 provide an internal support structure 201 integral with the chassis 100 (in particular the backsheet 26), that receives forces from the absorbent assembly 200 and transmits the forces toward the waist regions of the pull-on garment, specifically toward the closed side interfaces.

The absorbent core 250 can be disposed between a lower covering sheet that is disposed on the exterior face of the absorbent core 250 in a face-to-face arrangement with the interior surface 102 of the chassis and an upper covering sheet that is disposed on the interior face of the absorbent core 250. Such an upper covering sheet and lower covering sheet can be attached together to contain the absorbent core 250 between them and thereby form the absorbent assembly 200. For example, in the exemplary absorbent assembly 200 shown in FIGS. 23-25, an upper covering sheet 24 and a lower covering sheet 25 are attached together at or adjacent to the side edges 237a and 237b of the absorbent assembly 200 in longitudinally extending adhesive attachment zones 29a and 29b. Alternatively, the upper covering sheet 24 and the lower covering sheet 25 can be attached together in places other than the side edges 237a and 237b of the absorbent assembly 200, e.g., at or adjacent to the end edges 236 and 238 of the absorbent assembly 200, or at or adjacent to both the end edges 236 and 238 and the side edges 237a and 237b.

The upper covering sheet 24 is water-permeable and allows liquid waste to pass through to the absorbent core 250, where the liquid waste is absorbed. The lower covering sheet 25 can be water-impermeable. However, the lower covering sheet 25 preferably is water-permeable. In embodiments in which both the upper covering sheet 24 and the lower covering sheet 25 are water-permeable, any liquid waste that is deposited onto the upper covering sheet 24 but does not pass through the upper covering sheet 24 to the absorbent core 250 can flow around an edge of the absorbent assembly 200 to reach the lower covering sheet 25 and then pass through the lower covering sheet 25 to the absorbent core 250.

The upper covering sheet 24 can form the interior surface 202 of the absorbent assembly 200 that is intended to be placed against the body of the wearer. The upper covering sheet 24 preferably is formed of a soft material that will not irritate the skin of the wearer. Many materials that are suitable for a water-permeable covering sheet are well-known in the art, including synthetic nonwovens such as spunbonded or carded polypropylene, polyester, or rayon. Likewise, many materials that are suitable for a covering sheet that is water-impermeable are well-known in the art, including the materials that are suitable for the backsheet 26.

The upper covering sheet 24 and the lower covering sheet 25 can extend to the same width and the same length. Alternatively, one or more of the edges of one of the covering sheets can lie distally relative to the respective edge or edges of the other covering sheet. For example, the upper covering sheet can extend longitudinally only to an extent sufficient to cover the absorbent core and the lower covering sheet can extend longitudinally beyond the upper covering sheet toward or to the adjacent waist edge. Such an extended covering sheet can serve to isolate the skin of the wearer from a portion of the backsheet 26 as may be desirable, for example, when the garment 20 is worn under conditions in which contact between the skin and a backsheet film could be uncomfortable.

Suitable absorbent materials for the absorbent core 250 are well-known and can comprise any absorbent material that is generally compressible, conformable, non-irritating to the wearer's skin, and capable of absorbing and retaining liquids such as urine and other certain body exudates. The absorbent core 250 may comprise a wide variety of liquid-absorbent materials commonly used in disposable diapers and other absorbent articles such as comminuted wood pulp, which is generally referred to as air felt. Examples of other suitable absorbent materials include creped cellulose wadding; melt blown polymers, including co-form; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials. The absorbent core 250 can further comprise minor amounts (typically less than 10%) of non-liquid absorbent materials, such as adhesives, waxes, oils and the like. Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,209 (Dyer et al.); U.S. Pat. No. 5,397,316 (LaVon et al.); U.S. Pat. No. 5,625,222 (DesMarais et al.). These absorbent materials can be used separately or in combination. Many known absorbent materials can be used in a discrete form, i.e., in the form of fibers, granules, particles, and the like. Such a discrete form of an absorbent material can be immobilized by an adhesive that attaches the discrete pieces together to form a coherent layer or that attaches the discrete pieces to a substrate layer, such as a covering sheet, or that attaches the discrete pieces both to each other and to the substrate layer. Alternatively, the core 250 can comprise an absorbent polymer material in contact with a thermoplastic material. The absorbent polymer material can be further mixed with an absorbent fibrous material, such as airfelt material, or absorbent core 250 can be substantially airfelt free, as described in U.S. patent application Ser. No. 10/776,851 (Becker et al), published as U.S. Publication. No. 2004/0162536.

In the exemplary absorbent assembly 200 shown in FIGS. 23-25, the upper covering sheet 24 and the lower covering sheet 25 are of the same size, i.e., both the upper covering sheet 24 and the lower covering sheet 25 extend to the front edge 236 and back edge 238, as well as to the left side edge 237a and right side edge 237b of the absorbent assembly 200. Alternatively, the upper covering sheet 24 and the lower covering sheet 25 can differ in size. For example, the lower covering sheet 25 can be larger than the upper covering sheet 24 and can be wrapped over the side edges 257a and 257b of the absorbent core 250 onto the interior surface of the absorbent core 250, where the upper covering sheet 24 and the lower covering sheet 25 can be attached together. Alternatively, in place of a separate upper covering sheet 24 and a separate lower covering sheet 25, a single covering sheet can be wrapped around the absorbent core 250 and attached to itself to contain the absorbent core 250. Such a single covering sheet forms an upper layer and a lower layer when wrapped around the absorbent core 250 and, in general, the description of the separate upper covering sheet 24 and lower covering sheet 25 are intended to apply to such upper and lower layers of a wrapped single covering sheet.

At a minimum, the absorbent core 250 is contained laterally by the covering sheet or sheets being wrapped around the absorbent core 250 or attached together at or adjacent to the left side edge 237a and right side edge 237b of the absorbent assembly 200. For example, in the exemplary absorbent assembly 200 shown in FIGS. 23-25, the upper covering sheet 24 and the lower covering sheet 25 are attached together only in left adhesive attachment zone 29a and right adhesive attachment zone 29b at or adjacent to the respective left side edge 237a and right side edge 237b of the absorbent assembly 200. In this embodiment, the upper covering sheet 24 and the lower covering sheet 25 cannot be attached directly together at or adjacent to the front edge 236 and back edge 238 because the absorbent core 250 extends the full length of the absorbent assembly 200, i.e. the front edge 256 and back edge 258 of the absorbent core 250 coincide with the respective front edge 236 and back edge 238 of the absorbent assembly 200. In such an embodiment, the upper and lower layers of the covering sheet or sheets can each be attached to the absorbent core 250 at or adjacent to the front edge 256 and back edge 258 of the absorbent core 250 to form a sandwich. In addition, a sealing agent can be applied at or adjacent to the front edge 256 and back edge 258 of the absorbent core 250 to contain any fibers or particles that might otherwise escape the absorbent core 250. Alternatively, instead of being contained only laterally by the covering sheet or sheets, the absorbent core 250 can additionally be contained longitudinally by the upper and lower layers of the covering sheet or sheets being attached together at or adjacent to the front edge 236 and back edge 238 of the absorbent assembly 200.

Description of an Alternative Pull-On Garment

It should be appreciated that the present invention is not limited to the above, or any, particular chassis configuration, and that any chassis configuration that could benefit from the fit enhancement provided by the present invention is contemplated by the present invention.

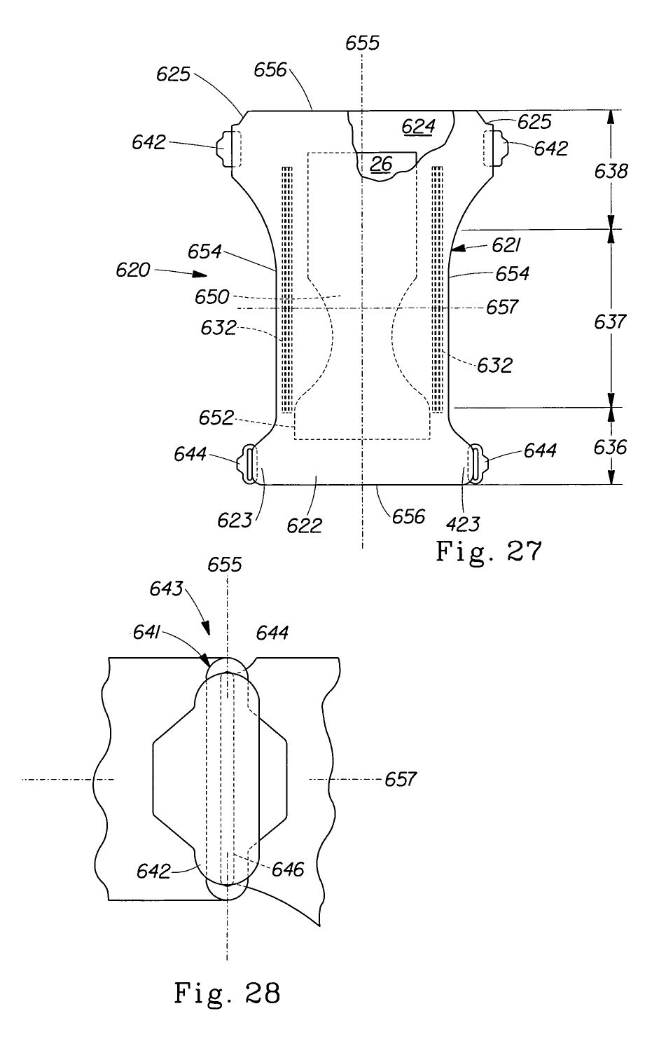

One such alternative chassis configuration is illustrated in FIG. 27. Specifically, a disposable absorbent article 620 constructed in accordance with an alternative embodiment includes a chassis 621 extending along a longitudinal axis or centerline 655 and a lateral axis or centerline 657. The garment chassis 621 includes a front waist region 636, a back waist region 638, and a crotch region 637 disposed between the waist regions. The waist regions 636 and 638 generally comprise those portions of the absorbent article 620 which, when worn, encircle the waist of the wearer. The crotch region 637 is that portion of the absorbent article 620 which, when the absorbent article 620 is worn, is generally positioned between the legs of the wearer. The chassis 621 defines an inner surface 650 that generally includes that portion of the absorbent article 620 which is positioned adjacent the wearer's body during use, and an outer surface 652 facing opposite the inner surface 650 and that generally comprises that portion of the absorbent article 620 which is positioned away from the wearer's body.

The outer periphery of the chassis 621 is defined by opposing laterally extending and longitudinally opposed end edges 656 that can be oriented generally parallel to the lateral axis 457, and by opposing longitudinally extending side edges 654 that can be oriented generally parallel to the longitudinal chassis axis 655 or, for better fit, can be curved or angled to produce an “hourglass” shape diaper when viewed in a plan view. Longitudinal chassis axis 655 bisects the end edges 656 while the lateral chassis axis 657 bisects the longitudinal edges 654.

The chassis 621 can comprises a liquid pervious topsheet 622, a liquid impervious backsheet 624, and an absorbent core 626 positioned between the topsheet 622 and the backsheet 624. The absorbent core 626 can have a body-facing surface and a garment facing-surface. The topsheet 622 can be disposed adjacent the body-facing surface of the absorbent core 626, while the backsheet 624 can be disposed adjacent the garment-facing surface of the absorbent core 626. It should be appreciated that the topsheet 622 can be attached to the core 626 and/or the backsheet 624 and that the backsheet 624 can be attached to the core 626 and/or the topsheet 622. It should be recognized that other structures, elements, or substrates can be positioned between the core 626 and the topsheet 622 and/or backsheet 624. In certain embodiments, the chassis 621 comprises the main structure of the absorbent article 620 with other features added to form the composite diaper structure. While the topsheet 622, the backsheet 624, and the absorbent core 626 can be assembled in a variety of well-known configurations, certain diaper configurations are described generally in U.S. Pat. Nos. 3,860,003; 5,151,092; 5,221,274; 5,554,145; 5,569,234; 5,580,411; and 6,004,306. The topsheet 622, the backsheet 624, and the absorbent core 626 are discussed in more detail below.

The absorbent article 620 can include front side panels 623 and back side panels 625, which can be unitary elements of the absorbent article 620 (i.e., they are not separately manipulative elements secured to the absorbent article 620, but rather are formed from and are extensions of one or more of the various layers of the diaper). In certain embodiments, the front and/or back side panels 623, 625 can be integral with the chassis 621 as shown in FIG. 27, or be discrete elements that are attached to the chassis 621. The front side panels 623 and back side panels 625 can be extensible, non-extensible, elastic, or ineleastic, and can be formed from nonwoven webs, woven webs, knitted fabrics, polymeric and elastomeric films, apertured films, sponges, foams, scrims, and combinations and laminates thereof. In certain embodiments the front side panels 623 and back side panels 625 can be formed of a nonwoven/elastomeric film laminate or a nonwoven/elastomeric film/nonwoven laminate. A suitable elastic side panel 623, 625 can be a laminate comprising an elastomeric film (such as is available from Tredegar Corp, Richmond, Va., as supplier code X25007) disposed between two nonwoven layers (such as is available from BBA Fiberweb, Brentwood, Tenn. under supplier code FPN332.

As described above, the topsheet 622 is generally a portion of the absorbent article 620 that can be positioned at least in partial contact or close proximity to a wearer. Accordingly, the topsheet 622 can be supple, soft feeling, and non-irritating to a wearer's skin. Generally, at least a portion of the topsheet 622 is liquid pervious, permitting liquids (e.g., urine) to readily penetrate through its thickness. The topsheet 622 can be made of a hydrophobic material to isolate the wearer's skin from liquids contained in the absorbent core 626. A portion of the topsheet 622 may be treated with a surfactant to render the topsheet permeable to liquid, or otherwise enhance the liquid permeability of the topsheet 622. Suitable topsheets 622 can be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. A suitable topsheet 622 is available from BBA Fiberweb, Brentwood, Tenn. as supplier code 055SLPV09U. Other examples of suitable topsheets 622 are described in U.S. Pat. No. 3,929,135, issued to Thompson on Dec. 30, 1975; U.S. Pat. No. 4,324,246 issued to Mullane et al. on Apr. 13, 1982; U.S. Pat. No. 4,342,314 issued to Radel et al on Aug. 3, 1982; U.S. Pat. No. 4,463,045 issued to Ahr et al. on Jul. 31, 1984; and U.S. Pat. No. 5,006,394 issued to Baird on Apr. 9, 1991.

Any portion of the topsheet 622 can be coated with a lotion as is known in the art. Examples of suitable lotions include those described in U.S. Pat. Nos. 5,607,760; 5,609,587; 5,635,191; and 5,643,588. The topsheet 622 can be fully or partially elasticized or can be foreshortened so as to provide a void space between the topsheet 622 and the core 626. Exemplary structures including elasticized or foreshortened topsheets are described in more detail in U.S. Pat. Nos. 4,892,536; 4,990,147; 5,037,416; and 5,269,775.

The absorbent core 626 generally is disposed between the topsheet 622 and the backsheet 624. The absorbent core 626 typically comprises a storage layer, which can be partially or totally surrounded by a core wrap. The storage layer can comprise any absorbent material that is generally compressible, conformable, non-irritating to the wearer's skin, and capable of absorbing and retaining liquids such as urine and other certain body exudates. The storage layer can comprise a wide variety of liquid-absorbent materials commonly used in disposable diapers and other absorbent articles such as comminuted wood pulp, which is generally referred to as air felt or fluff. Examples of other suitable absorbent materials include creped cellulose wadding; melt blown polymers, including co-form; chemically stiffened, modified or cross-linked cellulosic fibers; tissue, including tissue wraps and tissue laminates, absorbent foams, absorbent sponges, superabsorbent polymers (such as superabsorbent fibers), absorbent gelling materials, or any other known absorbent material or combinations of materials. Examples of some combinations of suitable absorbent materials are fluff with absorbent gelling materials and/or superabsorbent polymers, and absorbent gelling materials and superabsorbent fibers etc. In one optional embodiment the storage layer is air felt free, that is, it contains no air felt. The storage layer can further comprise minor amounts (typically less than 10%) of non-liquid absorbent materials, such as adhesives, waxes, oils and the like.

Exemplary absorbent structures for use as the absorbent assemblies are described in U.S. Pat. No. 4,834,735, entitled “High Density Absorbent Members Having Lower Density and Lower Basis Weight Acquisition Zones”, issued to Alemany et al. on May 30, 1989; and U.S. Pat. No. 5,625,222 entitled “Absorbent Foam Materials For Aqueous Fluids Made From high Internal Phase Emulsions Having Very High Water-To-Oil Ratios” issued to DesMarais et al. on Jul. 22, 1997.

In one optional embodiment of the present invention the absorbent core 626 comprises, in addition to the storage layer and the durable hydrophilic core wrap, an acquisition system, which comprises an upper acquisition layer facing towards the wearer and a lower acquisition layer. In one embodiment the upper acquisition layer comprises a nonwoven fabric whereas the lower acquisition layer comprises a mixture of chemically stiffened, twisted and curled fibers, high surface area fibers and thermoplastic binding fibers. In another embodiment both acquisition layers are provided from a non-woven material, which can be hydrophilic. The acquisition layer is in direct contact with the storage layer. Furthermore, the storage layer or parts thereof, such as the upper acquisition layer, can optionally be coated with the hydrophilicity boosting composition.

The backsheet 624 can be impervious to liquids (e.g., urine) and manufactured from a thin plastic film or a nonwoven web, although other flexible liquid impervious materials which are compliant and will readily conform to the general shape and contours of the human body can also be used. The backsheet 624 is generally positioned such that it can be at least a portion of the garment-facing surface of the absorbent article 620. The backsheet 624 prevents the exudates absorbed and contained therein from soiling articles that can contact the absorbent article 620, such as bed sheets and undergarments. Suitable backsheet 624 materials include films such as those manufactured by Tredegar Industries Inc. of Terre Haute, N and sold under the trade names X15306, X10962, and X10964. The backsheet can be a thermoplastic film having a thickness of from about 0.012 mm (0.5 mil) to about 0.051 mm (2.0 mils).

Other suitable backsheet 624 materials can include breathable materials that permit vapors to escape from the absorbent article 620 while still preventing exudates from passing through the backsheet 624. Exemplary breathable materials can include materials such as woven webs, nonwoven webs, polymeric films such as thermoplastic films of polyethylene or polypropylene, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746 and U.S. Pat. No. 5,865,823. Other breathable backsheets including nonwoven webs and apertured formed films are described in U.S. Pat. No. 5,571,096. An exemplary, suitable backsheet is disclosed in U.S. Pat. No. 6,107,537.

In one embodiment, the backsheet 626 can comprise a structural elastic-like film (SELF) web. SELF webs suitable for the present invention are more completely described in the commonly assigned U.S. Pat. No. 5,518,801 entitled “Web Materials Exhibiting Elastic-Like Behavior” issued to Chappell et al. on May 21, 1996. Other suitable materials and/or manufacturing techniques can be used to provide a suitable backsheet 624 including, but not limited to, surface treatments, particular film selections and processing, particular filament selections and processing, etc. The backsheet 424 can be embossed and/or matte finished to provide a more clothlike appearance. It should thus be appreciated that the backsheet 626 can be rendered extensible, or can be substantially inextensible as described above with respect to absorbent article 620.

FIG. 27 shows an embodiment of the absorbent article 620 in which the topsheet 622 and the backsheet 624 have length and width dimensions generally larger than those of the absorbent core 626. The topsheet 622 and the backsheet 624 extend beyond the edges of the absorbent core 626 to thereby form the periphery of the absorbent article 620. While the topsheet 622, the backsheet 624, and the absorbent core 626 can include many different materials and can be assembled in a variety of well known configurations, suitable diaper materials and configurations are described generally in U.S. Pat. No. 3,860,003 entitled “Contractable Side Portions for Disposable Diaper” which issued to Kenneth B. Buell on Jan. 14, 1975; and U.S. Pat. No. 5,151,092 issued to Buell on Sep. 9, 1992; and U.S. Pat. No. 5,221,274 issued to Buell on Jun. 22, 1993.

The absorbent article 620 can further include a pair of opposing and longitudinally extending leg cuffs 632 to improve containment of liquids and other body exudates. Each elasticized leg cuff 632 can include several different embodiments for reducing the leakage of body exudates in the leg regions. U.S. Pat. No. 3,860,003 describes a disposable diaper which provides a contractible leg opening having a side flap and one or more elastic members to provide an elasticized leg cuff (gasketing cuff). U.S. Pat. No. 4,909,803 entitled “Disposable Absorbent Article Having Elasticized Flaps” issued to Aziz et al. on Mar. 20, 1990, describes a disposable diaper having “stand-up” elasticized flaps (leg cuffs) to improve the containment of the leg regions. U.S. Pat. No. 4,695,278 entitled “Absorbent Article Having Dual Cuffs” issued to Lawson on Sep. 22, 1987, describes a disposable diaper having dual cuffs including a gasketing cuff and a leg cuff 632.

Referring also to FIG. 28, and as described above, each side edge 654 at the front waist region 636 is attached to itself at the back waist region 638 to define closed side interfaces 643 (one interface 643 illustrated in FIG. 28) and define a waist opening and a pair of leg openings as described above. The interfaces 643 can be closed using any fastening device such as an adhesive, cohesive, hooks-and-loops, or any alternative suitable fastening device. The absorbent article 620 can thus be pre-closed or pre-formed as described above. Alternatively, the absorbent article 620 can be configured as a fastenable diaper that is applied to the wearer before the left and right side edges 654 are closed using any suitable closing member to form the closed side interfaces 643 and secure the absorbent article 620 on the wearer (e.g., as in conventional taped diapers). The absorbent article 620, as illustrated, includes a fastening device 641 which attaches at least a portion of the front waist region 636 of the absorbent article 620 with at least a portion of the rear waist region 638 to form leg and waist openings.

The fastening device 641 comprises a first fastening element, such as a tab member 624, and a second fastening element such as a slot member 644. A “tab member” is broadly defined herein as an attachment member, at least a portion of which is configured to be passed through a mating slot member 644 to provide a fastened connection, while a “slot member” is broadly defined herein as an attachment member configured to receive at least a portion of a tab member to provide a fastened connection.

The tab members 642 can be positioned at the rear waist region 638 as illustrated, or at the front waist region 636, or at any alternative desired location such that the tab member 642 can readily connect to the slot member 644 to form the waist and leg openings.

During operation, the fastening device 641 is releasably and refastenably fastened by passing the tab member 642 through an elongated slot 446 of the slot member 644. The tab member 642 is then rotated into a plane generally parallel with the plane of the slot member 644 such that the tab member 642 overlaps at least a part of the slot member 644 to prevent the tab member 642 from slipping back through the slot 646 and disengaging the fastening device 641.

If desired, the tab member 642 can be disconnected from the slot member 644 by pulling the tab member 642 by aligning an edge of the tab member 642 with the slot 646, and pulling the tab member 642 through the slot 646.

All documents cited in the Detailed Description of the Invention are, in relevant part, incorporated herein by reference, however the citation of any document is not construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

1. A disposable absorbent article comprising:
a chassis defining a front waist region, a back waist region, a crotch region disposed between the waist regions, a waist opening, and a pair of leg openings;
an absorbent assembly attached to the chassis; and
a belt member attached to the chassis, the belt member extending across at least a portion of the chassis, the belt member including a frangible joint that is breakable to define a first and second free end, wherein the free ends are fastenable to at least one of each other and the chassis to impart a contractive force onto the chassis.

2. The disposable absorbent article as recited in claim 1, wherein the belt member is disposed in both the front and back waist regions of the garment.

3. The disposable absorbent article as recited in claim 1, wherein the belt member surrounds only a portion of the chassis before the frangible joint is broken.

4. The disposable absorbent article as recited in claim 3, wherein the frangible joint divides the belt member into first and second segments, each segment defining the first and second free ends, respectively.

5. The disposable absorbent article as recited in claim 1, wherein the belt further comprises an attachment zone disposed adjacent at least one of the free ends.

6. The disposable absorbent article as recited in claim 1, wherein at least one of the free ends is releasable and subsequently attachable to at least one of the chassis and a remaining segment of the belt member.

7. The disposable absorbent article as recited in claim 6, wherein the free ends are tied in a knot.

8. The disposable absorbent article as recited in claim 1, wherein the belt extends substantially along a longitudinal belt axis.

9. The disposable absorbent article as recited in claim 8, wherein at least a portion of the frangible joint extends in a direction parallel to the longitudinal belt axis.

10. The disposable absorbent article as recited in claim 9, wherein the frangible joint extends from opposing closed side interfaces on the chassis.

11. The disposable absorbent article as recited in claim 8, wherein at least a portion of the frangible joint extends in a direction perpendicular to the longitudinal belt axis.

12. The disposable absorbent article as recited in claim 8, wherein at least a portion of the frangible joint extends in a direction angled with respect to both 1) a direction perpendicular to the longitudinal belt axis 2) a direction parallel to the longitudinal belt axis.

13. The disposable absorbent article as recited in claim 1, wherein the belt member overlaps at least a portion of the front waist region.

14. The disposable absorbent article as recited in claim 13, wherein the belt member overlaps substantially all of the front waist region.

15. The disposable absorbent article as recited in claim 1, wherein the belt member overlaps at least a portion of the back waist region.

16. The disposable absorbent article as recited in claim 1, wherein the belt member overlaps substantially all of the front waist region.

17. The disposable absorbent article as recited in claim 1, wherein the belt member overlaps a closed side interface of the chassis.

18. The disposable absorbent article as recited in claim 1, wherein at least a portion of the belt member is extensible.

19. The disposable absorbent article as recited in claim 18, wherein the belt member is pre-tensioned.

20. The disposable absorbent article as recited in claim 18, wherein the belt member is non-pre-tensioned.

21. The disposable absorbent article as recited in claim 1, wherein at least a portion of the belt member is non-extensible.

22. The disposable absorbent article as recited in claim 1, wherein at least a portion of the chassis is extensible.

23. The disposable absorbent article as recited in claim 1, wherein at least a portion of the chassis is non-extensible.

24. The disposable absorbent article as recited in claim 1, further comprising a pull-on garment.

25. A method for securing a disposable absorbent article on a waist region of a wearer, the garment including a chassis defining a front waist region, a back waist region, and a crotch region disposed between the waist regions, the front and waist regions being closed to define a waist opening, the diaper further comprising a belt member attached to the chassis, the belt member including a frangible joint, wherein the method comprises the steps of:
A) pulling the absorbent article up to the waist region of the wearer such that the waist region is surrounded by the waist opening;
B) breaking the frangible joint to define first and second free ends;
C) after step (B), attaching at least one of the free ends to one of the chassis and a remaining portion of the belt member, thereby creating a contractive belt force to the chassis.

26. The method as recited in claim 25, wherein step (C) further comprising drawing the free ends together.

27. A disposable absorbent article comprising:
a chassis defining a front waist region, a back waist region, a crotch region disposed between the waist regions, a waist opening, and a pair of leg openings;
an absorbent assembly attached to the chassis; and
a belt member attached to the chassis at a first location, the belt member extending across at least portion of the chassis, the belt member defining opposing outer ends attached to the chassis at corresponding attachment zones, wherein at least one of the attachment zones is releasably attached to the chassis, wherein the releasable attachment zone can be released from the chassis and reattached to the chassis in a second location different than the first location, such that the belt applies a contractive belt force to the chassis.

28. The disposable absorbent article as recited in claim 27, further comprising a pull-on pant.
